SEQUENCE LISTING (20 sequences)

SEQ ID NO: 1 - DNA, 2433 bp, Homo sapiens
atggaacacaaagaagtggtgttgctcctgctgctgttcctgaagtccggccagggcgagcccctggacgattacgtgaacacccagggcgccagcctgttcagcgtgaccaagaaacagctgggagccggcagcatcgaggaatgcgccgccaagtgcgaagaggacgaggaattcacctgtcgggccttccagtaccacagcaaagaacagcagtgcgtgatcatggccgagaacagaaagagcagcatcatcatcagaatgcgggacgtggtgctgttcgagaagaaggtgtacctgagcgagtgcaagaccggcaacggcaagaactaccggggcaccatgagcaagaccaagaacggcatcacctgtcagaagtggtccagcaccagcccccaccggcctagattttctccagccacccaccctagcgagggcctggaagagaactactgccggaaccccgacaacgaccctcagggcccttggtgctacaccaccgaccccgagaagagatacgactactgcgacatcctggaatgtgaagaggaatgcatgcactgcagcggcgagaactacgacggcaagatctccaagaccatgagcggcctggaatgccaggcttgggacagccagtctcctcacgcccacggctacatccccagcaagttccccaacaagaacctgaagaagaattactgcagaaaccctgaccgcgagctgcggccctggtgttttaccaccgatcctaacaagagatgggagctgtgcgatatcccccggtgcaccacacctccacctagcagcggccctacctaccagtgtctgaagggcaccggcgagaattacaggggcaacgtggccgtgaccgtgtccggccatacctgccagcattggagcgcccagaccccccacacccacaacagaacccccgagaacttcccctgcaagaatctggacgagaattattgtcgcaaccccgatggcaagagggccccctggtgtcacaccaccaacagccaggtgcgctgggagtactgcaagatccccagctgcgatagcagccccgtgtccacagaacagctggcccctacagcccctcctgagctgacacctgtggtgcaggattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 2 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 3 - DNA, 1209 bp, Artificial Sequence
  recombinant PAI-1 Q197C and G355C
atgcagatgtctcccgccctgacctgcctggtgctgggcctggccctggtgttcggagagggctctgccgtgcaccacccacctagctacgtggcacacctggcctccgacttcggcgtgagggtgtttcagcaggtggcccaggccagcaaggatcgcaacgtggtgttcagcccttatggcgtggcctccgtgctggccatgctccagctgaccacaggaggagagacccagcagcagatccaggcagctatgggcttcaagatcgacgataagggaatggcacccgccctgaggcacctgtacaaggagctgatgggcccttggaataaggacgagatcagcaccacagatgccatctttgtgcagcgcgacctgaagctggtgcagggcttcatgccacacttctttcggctgttccggagcaccgtgaagcaggtggacttcagcgaggtggagagggcccgctttatcatcaacgattgggtgaagacccacacaaagggcatgatcagcaatctgctgggcaagggagcagtggatcagctgaccaggctggtgctggtgaacgccctgtacttcaatggctgctggaagaccccatttcccgacagctccacacaccggagactgttccacaagtccgatggctctacagtgagcgtgcctatgatggcccagaccaacaagttcaattatacagagtttaccacacctgacggccactactatgacatcctggagctgccataccacggcgacaccctgagcatgtttatcgccgccccttatgagaaggaggtgccactgtccgccctgacaaacatcctgtccgcccagctgatctctcactggaagggcaatatgaccaggctgccaaggctgctggtgctgcctaagttctccctggagacagaggtggacctgcggaagcctctggagaacctgggcatgaccgatatgttcagacagtttcaggccgactttacatctctgagcgatcaggagccactgcacgtggcacaggccctccagaaggtgaagatcgaggtgaacgagtcctgtaccgtggcctctagctccacagccgtgatcgtgtctgccaggatggccccagaggagatcatcatggatcggcccttcctgtttgtggtgagacacaatccaaccggcacagtgctgttcatgggccaggtcatggagccctga

SEQ ID NO: 4 - protein, 402 aa, Artificial Sequence
  recombinant PAI-1, Q197C, G355C
MQMSPALTCLVLGLALVFGEGSAVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISNLLGKGAVDQLTRLVLVNALYFNGCWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVAQALQKVKIEVNESCTVASSSTAVIVSARMAPEEIIMDRPFLFVVRHNPTGTVLFMGQVMEP

SEQ ID NO: 5 - DNA, 4241 bp, Artificial Sequence
  hPlg-IRES2-PAI-1 expression cassette
atggaacacaaagaagtggtgttgctcctgctgctgttcctgaagtccggccagggcgagcccctggacgattacgtgaacacccagggcgccagcctgttcagcgtgaccaagaaacagctgggagccggcagcatcgaggaatgcgccgccaagtgcgaagaggacgaggaattcacctgtcgggccttccagtaccacagcaaagaacagcagtgcgtgatcatggccgagaacagaaagagcagcatcatcatcagaatgcgggacgtggtgctgttcgagaagaaggtgtacctgagcgagtgcaagaccggcaacggcaagaactaccggggcaccatgagcaagaccaagaacggcatcacctgtcagaagtggtccagcaccagcccccaccggcctagattttctccagccacccaccctagcgagggcctggaagagaactactgccggaaccccgacaacgaccctcagggcccttggtgctacaccaccgaccccgagaagagatacgactactgcgacatcctggaatgtgaagaggaatgcatgcactgcagcggcgagaactacgacggcaagatctccaagaccatgagcggcctggaatgccaggcttgggacagccagtctcctcacgcccacggctacatccccagcaagttccccaacaagaacctgaagaagaattactgcagaaaccctgaccgcgagctgcggccctggtgttttaccaccgatcctaacaagagatgggagctgtgcgatatcccccggtgcaccacacctccacctagcagcggccctacctaccagtgtctgaagggcaccggcgagaattacaggggcaacgtggccgtgaccgtgtccggccatacctgccagcattggagcgcccagaccccccacacccacaacagaacccccgagaacttcccctgcaagaatctggacgagaattattgtcgcaaccccgatggcaagagggccccctggtgtcacaccaccaacagccaggtgcgctgggagtactgcaagatccccagctgcgatagcagccccgtgtccacagaacagctggcccctacagcccctcctgagctgacacctgtggtgcaggattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactgaaagcttggtaccgagctcggatcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatgcagatgtctcccgccctgacctgcctggtgctgggcctggccctggtgttcggagagggctctgccgtgcaccacccacctagctacgtggcacacctggcctccgacttcggcgtgagggtgtttcagcaggtggcccaggccagcaaggatcgcaacgtggtgttcagcccttatggcgtggcctccgtgctggccatgctccagctgaccacaggaggagagacccagcagcagatccaggcagctatgggcttcaagatcgacgataagggaatggcacccgccctgaggcacctgtacaaggagctgatgggcccttggaataaggacgagatcagcaccacagatgccatctttgtgcagcgcgacctgaagctggtgcagggcttcatgccacacttctttcggctgttccggagcaccgtgaagcaggtggacttcagcgaggtggagagggcccgctttatcatcaacgattgggtgaagacccacacaaagggcatgatcagcaatctgctgggcaagggagcagtggatcagctgaccaggctggtgctggtgaacgccctgtacttcaatggctgctggaagaccccatttcccgacagctccacacaccggagactgttccacaagtccgatggctctacagtgagcgtgcctatgatggcccagaccaacaagttcaattatacagagtttaccacacctgacggccactactatgacatcctggagctgccataccacggcgacaccctgagcatgtttatcgccgccccttatgagaaggaggtgccactgtccgccctgacaaacatcctgtccgcccagctgatctctcactggaagggcaatatgaccaggctgccaaggctgctggtgctgcctaagttctccctggagacagaggtggacctgcggaagcctctggagaacctgggcatgaccgatatgttcagacagtttcaggccgactttacatctctgagcgatcaggagccactgcacgtggcacaggccctccagaaggtgaagatcgaggtgaacgagtcctgtaccgtggcctctagctccacagccgtgatcgtgtctgccaggatggccccagaggagatcatcatggatcggcccttcctgtttgtggtgagacacaatccaaccggcacagtgctgttcatgggccaggtcatggagccctga

SEQ ID NO: 6 - DNA, 2433 bp, Homo sapiens; identical to SEQ ID NO: 1

SEQ ID NO: 7 - protein, 810 aa, Homo sapiens; identical to SEQ ID NO: 2

SEQ ID NO: 8 - DNA, 2202 bp, Homo sapiens
atggaacacaaagaagtggtgttgctcctgctgctgttcctgaagtccggccagggcaaggtgtacctgagcgagtgcaagaccggcaacggcaagaactaccggggcaccatgagcaagaccaagaacggcatcacctgtcagaagtggtccagcaccagcccccaccggcctagattttctccagccacccaccctagcgagggcctggaagagaactactgccggaaccccgacaacgaccctcagggcccttggtgctacaccaccgaccccgagaagagatacgactactgcgacatcctggaatgtgaagaggaatgcatgcactgcagcggcgagaactacgacggcaagatctccaagaccatgagcggcctggaatgccaggcttgggacagccagtctcctcacgcccacggctacatccccagcaagttccccaacaagaacctgaagaagaattactgcagaaaccctgaccgcgagctgcggccctggtgttttaccaccgatcctaacaagagatgggagctgtgcgatatcccccggtgcaccacacctccacctagcagcggccctacctaccagtgtctgaagggcaccggcgagaattacaggggcaacgtggccgtgaccgtgtccggccatacctgccagcattggagcgcccagaccccccacacccacaacagaacccccgagaacttcccctgcaagaatctggacgagaattattgtcgcaaccccgatggcaagagggccccctggtgtcacaccaccaacagccaggtgcgctgggagtactgcaagatccccagctgcgatagcagccccgtgtccacagaacagctggcccctacagcccctcctgagctgacacctgtggtgcaggattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 9 - protein, 733 aa, Homo sapiens
MEHKEVVLLLLLFLKSGQGKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 10 - DNA, 1365 bp, Homo sapiens
atggaacacaaagaagtggtgttgctcctgctgctgttcctgaagtccggccagggcgattgctaccacggcgacggccagagctacagaggcaccagcagcaccaccacaaccggcaagaagtgccagagctggtcctccatgacccctcaccggcaccagaaaacccctgagaattaccccaacgccggcctgaccatgaactactgtagaaatcccgacgccgacaagggaccctggtgcttcacaacagacccttccgtcagatgggaatactgtaatctgaagaagtgcagcggcaccgaggccagcgtggtggctcctccaccagtggtgctgctgcccgatgtggaaaccccctccgaagaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 11 - protein, 454 aa, Homo sapiens
MEHKEVVLLLLLFLKSGQGDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 12 - DNA, 1056 bp, Homo sapiens
atggaacacaaagaagtggtgttgctcctgctgctgttcctgaagtccggccagggcgaggactgtatgttcggcaatggcaagggctatagaggcaagcgggccaccaccgtgaccggcacaccttgtcaggattgggccgctcaggaaccccacagacacagcatcttcaccccagagacaaaccctcgggccggactggaaaaaaactattgtcggaatcctgacggcgacgtgggaggaccttggtgttatacaacaaacccacggaagctgtacgattactgtgacgtgccccagtgtgccgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 13 - protein, 351 aa, Homo sapiens
MEHKEVVLLLLLFLKSGQGEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 14 - DNA, 807 bp, Homo sapiens
atggaacacaaagaagtggtgttgctcctgctgctgttcctgaagtccggccagggcgcccctagcttcgattgtggcaagccccaggtggaacccaagaaatgccccggcagagtcgtgggcggatgtgtggcccatcctcactcttggccttggcaggtgtccctgcggaccagattcggcatgcacttttgcggcggcaccctgatcagccccgagtgggtgctgacagccgcccactgtctggaaaagtcccccagacccagcagctacaaagtgatcctgggagcccaccaggaagtgaacctggaacctcacgtgcaggaaatcgaggtgtccagactgttcctggaacccacccggaaggatatcgccctgctgaagctgagcagccctgccgtgatcaccgacaaagtgattcccgcctgcctgcccagccccaactatgtggtggccgacagaaccgagtgcttcatcaccggctggggcgagacacagggcacatttggagccggcctgctgaaagaggcccagctgcctgtgatcgagaacaaagtgtgcaaccgctacgagttcctgaacggcagagtgcagagcaccgagctgtgtgccggacatctggctggcggcacagatagctgtcagggcgattctggcggccctctcgtgtgcttcgagaaggacaagtacatcctgcagggcgtgaccagctggggcctgggatgtgccagacctaacaagcccggcgtgtacgtgcgcgtgtccagatttgtgacctggatcgagggcgtgatgcggaacaactga

SEQ ID NO: 15 - protein, 268 aa, Homo sapiens
MEHKEVVLLLLLFLKSGQGAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 16 - protein, 791 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 17 - protein, 714 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 18 - protein, 435 aa, Homo sapiens
DCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 19 - protein, 332 aa, Homo sapiens
EDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 20 - protein, 249 aa, Homo sapiens
APSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

(19) United States
(12) Patent Application Publication
(10) Pub. No.: US 2022/0267751 A1
(43) Pub. Date: Aug. 25, 2022

(54) METHODS FOR MAKING RECOMBINANT PROTEIN

(71) Applicant: Monash University, Clayton (AU)

(72) Inventors: James Whisstock, Victoria (AU); Ruby Law, Victoria (AU); Adam Quek, Victoria (AU); Paul Conroy, Victoria (AU)

(73) Assignee: Monash University, Clayton (AU)

(21) Appl. No.: 17/626,168

(22) PCT Filed: Jul. 10, 2020
(86) PCT No.: PCT/AU2020/050719

§ 371 Date: Jan. 11, 2022

(30) Foreign Application Priority Data
Jul. 12, 2019 (AU) 2019902468

(51) Int. Cl. C12N 9/68; C07K 14/81

(52) U.S. Cl. CPC C12N 9/6435; C12Y 304/21007; C07K 14/8132

(57) ABSTRACT

The present invention relates to methods of producing recombinant plasminogen in a mammalian expression system. A method for producing plasminogen, the method comprising (i) providing a host cell comprising a first recombinant polynucleotide encoding plasminogen and a second recombinant polynucleotide encoding a plasminogen activation inhibitor; (ii) culturing said host cell in a suitable culture medium under conditions to effect expression of plasminogen from the first polynucleotide and plasminogen activation inhibitor from the second polynucleotide.

### RELATED APPLICATIONS

This Application is a national stage filing under 35 U.S.C. 371 of International Patent Application Serial No. PCT/AU2020/050719, filed Jul. 10, 2020. Foreign priority benefits are claimed under 35 U.S.C. § 119(a)-(d) or 35 U.S.C. § 365(b) of Australian application number 2019902468, filed Jul. 12, 2019. The entire contents of these applications are incorporated herein by reference in their entirety.

### FIELD OF THE INVENTION

The present invention relates to methods of producing recombinant plasminogen in a mammalian expression system.

### BACKGROUND OF THE INVENTION

The production of large quantities of relatively pure polypeptides and proteins is important for the manufacture of many pharmaceutical formulations. For production of many proteins, recombinant DNA techniques have been employed in part because large quantities of exogenous proteins can be expressed in host cells.

Plasmin is the principal fibrinolytic enzyme in mammals. This protein is a serine protease belongs to the chymotrypsin-like family that is derived from the inactive zymogen precursor plasminogen, circulating in plasma.

Plasminogen is a single-chain glycoprotein consisting of 791 amino acids with a molecular mass of approximately 92 kDa. Plasminogen is mainly synthesized in the liver and is abundant in most extracellular fluids. In plasma the concentration of plasminogen is approximately 2 μM. Plasminogen therefore constitutes a large potential source of proteolytic activity in tissues and body fluids.

Plasminogen exists in two molecular forms: Glu-plasminogen and Lys-plasminogen. The native secreted and uncleaved form has an amino-terminal (N-terminal) glutamic acid and is therefore designated Glu-plasminogen. However, in the presence of plasmin, Glu-plasminogen is cleaved at Lys76-Lys77 to become Lys-plasminogen. Compared to Glu-plasminogen, Lys-plasminogen has a higher affinity for fibrin and is activated by plasminogen activators at a higher rate, however, there is no evidence that Lys-plasminogen is found in the circulation.

Plasminogen is activated to plasmin by cleavage of the Arg561-Va1562 peptide bond by either tissue-type plasminogen activator (tPA) or urokinase-type plasminogen activator (uPA). This cleavage results in an α-heavy-chain consisting of one pan-apple and five kringle domains, four of these kringle domains with lysine-binding sites and a 13 light-chain with the catalytic triad, namely His603, Asp646, and Ser741. The active plasmin is involved in the lysis of fibrin clots in the host. It has been shown that, upon binding to a fibrin clot, the Pan-apple domain of the native plasminogen (with an N-terminal glutamic acid Glu-plasminogen) is readily cleaved and converted into a modified Plasminogen (83 kDa) with an N-terminal lysine (Lys-plasminogen).

Two major glycoforms of plasminogen exist in human plasma: Type 1 plasminogen, which contains at least two glycosylation moieties (N-linked to N289 and O-linked to T346), and Type 2 plasminogen, which contains at least one O-linked sugar (O-linked to T346). Type 2 plasminogen is preferentially recruited to the cell surface, whereas Type 1 plasminogen is more predominantly recruited to blood clots.

Plasminogen can exist in two conformations: closed and open. The native Glu-plasminogen in the circulation is in the closed form as such that the activation site is not exposed. Once bound to the target, such as fibrin clot or cell surface receptor, via the lysine-binding sites on the kringle domains, it changes to an open conformation with its activation site exposed. The dimensions of the molecule differ significantly between these two conformations.

Plasmin is a fundamental component of the fibrinolytic system and is the main enzyme involved in the lysis of blood clots and clearance of extravasated fibrin. In addition, plasmin cleaves a wide range of biological targets including basement membrane, extracellular matrices, cell receptors, cytokines and complements. Plasminogen is therefore vital in wound healing, cell migration, tissue remodeling, angiogenesis and embryogenesis. Plasminogen has been implicated in multiple cell processes during all phases of wound healing—inflammatory, proliferative, and remodeling. These processes include fibrin degradation, platelet activation, release of cytokines and growth factors, clearance of apoptotic cells, activation of keratinocytes and epithelial-to-mesenchymal transition of fibroblasts, cell migration, and extracellular matrix degradation.

There are numerous technical difficulties associated with obtaining sufficient quantities and sufficiently pure preparations of recombinant plasminogen for use a therapeutic agent. Because of the complex structure of the full-length plasminogen molecule, bacterial expression systems have not proven useful for recombinant plasminogen production. Plasminogen is produced in the form of insoluble inclusion bodies and is not re-foldable from that state. Further, the expression of plasminogen in mammalian cells is complicated by intracellular activation of plasminogen into plasmin and the resulting cytotoxicity. Production of fully active plasminogen using insect cells is possible, however, this system is not suitable for large-scale production due to low yield.

As a consequence of the difficulty in obtaining suitable amounts and quality of recombinant plasminogen using recombinant systems, most plasminogen produced for use in clinical settings today is derived from fractionation of plasma. Obtaining plasminogen directly from human plasma presents with its own problems including the need to rely on sufficient donations of source material and risks of pathogen contamination thereof.

There is need for new methods and compositions for obtaining plasminogen, in sufficient amounts and with sufficient activity, for use in treatment of conditions requiring plasminogen supplementation.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method for producing plasminogen, the method comprising:
(i) providing a host cell comprising a first recombinant polynucleotide encoding plasminogen and a second recombinant polynucleotide encoding a plasminogen activator inhibitor;
(ii) culturing said host cell in a suitable culture medium under conditions to effect expression of plasminogen from the first polynucleotide and plasminogen activator inhibitor from the second polynucleotide.

Preferably, the present invention provides a method for producing plasminogen, the method comprising:
(i) providing a host cell comprising a first recombinant polynucleotide encoding plasminogen and a second recombinant polynucleotide encoding plasminogen activator inhibitor-1 (PAI-1) or variant thereof;
(ii) culturing said host cell in a suitable culture medium under conditions to effect expression of plasminogen from the first polynucleotide and PAI-1 or variant thereof from the second polynucleotide.

In one aspect, the present invention provides a method of producing recombinant plasminogen, the method comprising the steps of:

(a) providing a first polynucleotide encoding plasminogen,

(b) providing a second polynucleotide encoding PAI-1 or variant thereof; wherein the first and the second polynucleotide are operably linked to a promoter for enabling the expression of the polynucleotides,

(c) providing a host cell,

(d) transforming or transfecting the host cell with the polynucleotides of a) and b)

(e) providing cell culture media,

(f) culturing the transformed or transfected host cell in the cell culture media under conditions sufficient for expression of the polynucleotides encoding plasminogen and the PAI-1 or variant thereof, and optionally (g) recovering or purifying plasminogen from the host cell and/or the cell culture media.

In one embodiment, the first and second polynucleotides are provided in a single polynucleotide molecule. In alternative embodiments, the first and second polynucleotides are provided in different polynucleotide molecules. For example, the polynucleotides encoding plasminogen and plasminogen activator inhibitor (preferably PAI-1) or variant thereof, may be provided in a single vector. Alternatively, the polynucleotides encoding plasminogen and plasminogen activator inhibitor (PAI-1) or variant thereof may be provided in separate vector constructs, each vector having a different type of selection marker from the other vector.

In another aspect, the present invention also provides a method of producing recombinant plasminogen, the method comprising the steps of:

(a) providing a vector comprising a polynucleotide encoding plasminogen,

(b) providing a vector comprising a polynucleotide encoding PAI-1 or variant thereof;

(c) providing a host cell,

(d) transforming or transfecting the host cell with the vector of steps (a) and (b),

(e) providing cell culture media,

(f) culturing the transformed or transfected host cell in the cell culture media under conditions sufficient for expression of the polynucleotides encoding plasminogen and the PAI-1 or variant thereof, and

optionally (g) recovering or purifying plasminogen from the host cell and/or the cell culture media.

In any aspect of a method of the invention, the method further comprises the step of admixing a PAI-1 or variant thereof into the culture media.

In another aspect, the present invention provides a method for producing plasminogen, the method comprising:
(i) providing a host cell comprising a recombinant polynucleotide encoding plasminogen;
(ii) culturing said host cell in a suitable culture medium under conditions to effect expression of plasminogen from the polynucleotide, wherein the culture media comprises PAI-1 or variant thereof.

In another aspect, the present invention provides a method of producing recombinant plasminogen, the method comprising the steps of:

(a) providing a polynucleotide encoding plasminogen,

wherein the polynucleotide is operably linked to a promoter for enabling the expression of the polynucleotide,

(c) providing a host cell,

(d) transforming or transfecting the host cell with the polynucleotide of (a)

(e) providing cell culture media comprising PAI-1 or variant thereof,

(f) culturing the transformed or transfected host cell in the cell culture media under conditions sufficient for expression of the polynucleotide encoding plasminogen, and

optionally (g) recovering or purifying plasminogen from the host cell and/or the cell culture media.

In any aspect of a method of the invention, the method provides for transient or stable expression of the polynucleotide encoding plasminogen and transient or stable expression of the polynucleotide encoding the PAI-1 or variant thereof.

The plasminogen may correspond to the plasminogen sequence of any mammal. In any embodiment, the plasminogen is human plasminogen, non-human primate plasminogen, pig, mouse, rat, sheep, goat, horse, cow, cat, dog, or other mammalian plasminogen. Preferably, the plasminogen is human plasminogen.

In any embodiment of the invention, the plasminogen is selected from the group consisting of: Glu-Plg, Lys-Plg, Midi-Plg, Mini-Plg and Micro-Plg.

The plasminogen may comprise the wild-type plasminogen sequence, or may comprise a variant or modified sequence thereof. In any embodiment of the invention, the plasminogen is selected from the group consisting of: Glu-Plg, Lys-Plg, Midi-Plg, Mini-Plg and Micro-Plg. In alternative embodiments, the plasminogen may comprise a plasminogen sequence comprising amino acid substitutions at the protease active site, at the activation site, and combinations thereof and or amino acid substitutions that lead to increased protease activity.

In certain embodiments, the polynucleotide encoding the plasminogen comprises, consists or consists essentially of the nucleic acid sequence of any one of SEQ ID NOs: 1, 5, 6, 8, 10, 12 or 14, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% sequence identity to the sequence set forth in any one of SEQ ID NOs: 1, 5, 6, 8, 10, 12 or 14.

In certain embodiments, the plasminogen encoded by the polynucleotide, or produced according to any method described herein, comprises, consists or consists essentially of an amino acid sequence as set forth in any one of SEQ ID NOs: 2, 7, 9, 11, 13, 15, 16, 17, 18, 19 or 20, or a sequence that is at least 75%, at least 80%, at least 85%, at least 90%, at least 91%, at least 92%, at least 93%, at least 94%, at least 95%, at least 96%, at least 97%, at least 98%, at least 99% identical to the amino acid sequence as set forth in any of SEQ ID NOs: 2, 7, 9, 11, 13, 15, 16, 17, 18, 19 or 20. In one embodiment, the plasminogen produced according to any method described herein does not contain a signal sequence, including any signal sequence described herein.

Preferably, the plasminogen activator inhibitor is PAI-1. More preferably, the PAI-1 comprises, consists, or consists essentially of the amino acid sequence as shown in SEQ ID NO: 4. Alternatively, the PAI-1 sequence may comprise, consist or consist essentially of the sequence for unmodified (i.e., wild-type) PAI-1, wherein the wild-type sequence consists of the sequence of SEQ ID NO: 4, wherein the residues at positions 197 and 355 are glutamine and glycine, respectively.

In certain embodiments, the polynucleotide encoding the plasminogen activator inhibitor comprises the nucleic acid sequence of SEQ ID NO: 3, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% identity to the sequence set forth in SEQ ID NO:3.

In certain embodiments, wherein the first and second polynucleotides (i.e., the polynucleotides encoding plasminogen and plasminogen activation inhibitor) are provided in a single polynucleotide construct, such as a single vector construct, the single polynucleotide construct comprises, consists of or consists essentially of the nucleic acid sequence as shown in SEQ ID NO: 5, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% sequence identity to the sequence set forth in SEQ ID: 5.

The host cell is preferably a mammalian host cell, including but not limited to a cell selected from the group consisting of: Expi293, variants of Expi293, CHO (Chinese Hamster Ovary) cells and derivatives thereof, HeLa (Human cervical cancer) cells, COS and Vero cells.

In another aspect, the present invention also provides a vector or construct comprising a first polynucleotide sequence encoding plasminogen and a second polynucleotide sequence encoding PAI-1 or variant thereof. Preferably, the first and the second polynucleotide are operably linked to a promoter for enabling the expression of the polynucleotides. In certain embodiments, the plasminogen and PAI-1 are encoded in a single polynucleotide construct to enable bicistronic expression. In certain embodiments, the vector or construct comprises an internal ribosome entry site (IRES) between the first polynucleotide sequence and second polynucleotide sequence that allows for translation initiation in a cap-independent manner.

Typically, the first polynucleotide sequence encodes a plasminogen selected from the group consisting of: Glu-Plg, Lys-Plg, Midi-Plg, Mini-Plg and Micro-Plg.

In certain embodiments, the first polynucleotide comprises, consists or consist essentially of the nucleic acid sequence as set forth in any one of SEQ ID NOs: 1, 6, 8, 10, 12 or 14 or a nucleic acid sequence having at least 75%, at least 80%, at least 85%, at least 90%, at least 91%, at least 92%, at least 93%, at least 94%, at least 95%, at least 96%, at least 97%, at least 98%, at least 99% sequence identity to the sequence set forth in any one of SEQ ID NOs: 1, 6, 8, 10, 12 or 14.

In certain embodiments, the second polynucleotide comprises, consists or consists essentially of the nucleic acid sequence of SEQ ID NO: 3, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% identity to the sequence set forth in SEQ ID NO:3.

In certain embodiments, the plasminogen encoded by the first polynucleotide comprises, consists or consists essentially of, or has an amino acid sequence that is at least 75%, at least 80%, at least 85%, at least 90%, at least 91%, at least 92%, at least 93%, at least 94%, at least 95%, at least 96%, at least 97%, at least 98%, at least 99% identical to the amino acid sequence set forth in any one of SEQ ID NOs: 2, 7, 9, 11, 13, 15, 16, 17, 18, 19 or 20.

In one embodiment, the plasminogen activation inhibitor encoded by the second polynucleotide is plasminogen activator inhibitor-1 (PAI-1) or variant thereof. More preferably, the PAI-1 comprises, consists, or consists essentially of the amino acid sequence as shown in SEQ ID NO: 4. Alternatively, the PAI-1 sequence may comprise, consist or consist essentially of the sequence for unmodified (i.e., wild-type) PAI-1, wherein the wild-type sequence consists of the sequence of SEQ ID NO: 4, wherein the residues at positions 197 and 355 are glutamine and glycine, respectively.

In one embodiment, the vector or construct comprises a nucleic acid sequence as shown in SEQ ID NO: 5, or a nucleic acid sequence having at least 75%, at least 80%, at least 85%, at least 90%, at least 91%, at least 92%, at least 93%, at least 94%, at least 95%, at least 96%, at least 97%, at least 98%, at least 99% sequence identity to the sequence set forth in SEQ ID: 5.

In another aspect, the present invention provides a host cell comprising a vector or construct of the invention as described herein.

In another aspect, the present invention provides isolated, purified, substantially purified or recombinant plasminogen produced by a method of the invention as described herein. The plasminogen may be any one described herein, for example may comprises, consists or consists essentially of an amino acid sequence as set forth in any one of SEQ ID NOs: 2, 7, 9, 11, 13, 15, 16, 17, 18, 19 or 20, or a sequence that is at least 75%, at least 80%, at least 85%, at least 90%, at least 91%, at least 92%, at least 93%, at least 94%, at least 95%, at least 96%, at least 97%, at least 98%, at least 99% identical to the amino acid sequence as set forth in any of SEQ ID NOs: 2, 7, 9, 11, 13, 15, 16, 17, 18, 19 or 20. Preferably, the plasminogen does not contain a signal sequence, including any signal sequence described herein.

In another aspect, the present invention provides a composition comprising plasminogen and plasminogen activator inhibitor, preferably PAI-1 or variant thereof, isolated, purified or substantially purified from the culture media from a method of the invention as described herein.

In a further aspect, the present invention provides isolated, purified, substantially purified, or recombinant plasmin derived or obtained from plasminogen that is produced by a method of the invention as described herein.

Still further, the present invention provides for the use of isolated, purified, substantially purified or recombinant plasminogen (or plasmin derived therefrom) in a method of treating a condition in an individual, wherein the condition requires administration of exogenous plasminogen (or plasmin).

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Coomassie stained 10% SDS-PAGE of protein fractions eluted from affinity column. Protein bands at around 100 kDa represent eluted rPlg.

FIG. 2: Anion exchange chromatography of rPlg. (A) Purification of rPlg (indicated by double-headed arrow) by anion exchange chromatography using a gradient of high salt buffer B (wherein rPlg is eluted as a single peak). Contaminants (indicated by “c”) are separated from affinity purified rPlg (see FIG. 1) and eluted later in the presence of 100% buffer B. (B) Coomassie-stained 10% SDS-PAGE showing fractions containing rPlg as indicated by the double-headed arrow in (A).

FIG. 3: Size exclusion chromatography of rPlg. (A) Size exclusion profile of rPlg purified on a Superdex 200 column. (B) Coomassie stained 12% SDS-PAGE showing the purified rPlg.

FIG. 4: Coomassie stained 12% SDS-PAGE showing recombinant plasmin (rPlm) generated by activation of rPlg using tPA. Under reducing conditions, rPlm is separated into a heavy and a light chain (residues Glu₁-Arg₅₆₁ and Val₅₆₂-Asn₇₉₁, respectively).

FIG. 5: Progress curve showing tPA-mediated activation of Plgs (500 nM). Activation of rPlg, native Plg glycoform I and II (Plg GI and Plg GII), measured by the hydrolysis of plasmin fluorogenic substrate H-Ala-Phe-Lys-AMC.

FIG. 6: Michelis-Menten analysis of Plg activation by tPA in the presence of 1 μM EACA. Results from activation of rPlg, native Plg GI, and GII, indicates that rPlg is most readily-activatable (as indicated by the K_{M} and Vₘₐₓ).

FIG. 7: Radius of gyration of native Plg GI, Gil and rPlg. Experiments were performed using Small Angle X-ray Scattering which measures the dimensions of macromolecules in solution. The closed and open conformations were recorded in the presence or absence of 20 mM EACA, respectively. In the closed form, the R_{g} of GI and rPlg are similar.

FIG. 8: SAXS titration experiments to study the conformational change of Plg in response to EACA. Overall, the titration curves are comparable amongst GI, GII and rPlg. Kₒₚₑₙ is the EACA concentration at which 50% of Plg is in the open conformation.

FIG. 9: Binding of rPlg and native Plg to α2-AP. Binding of Plg (at concentrations as shown) to α2-AP immobilised on a Ni²⁺⁻NTA chip was measured in real-time. Coloured lines represent experimental curves and dotted lines represent fitted curves. A two-state reaction model was used to calculate kinetics and affinity constants for binding of native Plg (bottom) and a 1:1 Langmuir binding model for binding of rPlg (top) using the Biacore T200 evaluation software (Biacore AB) and used to calculate kinetics and affinity constants.

FIG. 10: Binding of rPlm and native Plm to α2-AP. Binding of Plm (at concentrations as shown) to α2-AP immobilised on a Ni²⁺⁻NTA chip was measured in real-time. Coloured lines represent experimental curves and dotted lines represent fitted curves. The data were fitted with a 1:1 Langmuir binding model using the Biacore T200 evaluation software (Biacore AB) and used to calculate kinetics and affinity constants.

FIG. 11: Binding of rPlg and native Plg to streptokinase (SK). Binding of recombinant plasminogen and native plasminogen to SK immobilised on Ni²⁺⁻NTA chip, measured in real-time. Coloured lines represent experimental curves and dotted lines represent fitted curves. The data were fitted with a 1:1 Langmuir binding model using the Biacore T200 evaluation software (Biacore AB) and used to calculate kinetics and affinity constants.

FIG. 12: Binding of rPlm and native Plm to SK. Binding of recombinant plasmin and native plasmin to streptokinase immobilised on Ni²⁺⁻NTA chip, measured in real-time. Coloured lines represent experimental curves and dotted lines represent fitted curves. The data were fitted with a 1:1 Langmuir binding model using the Biacore T200 evaluation software (Biacore AB) and used to calculate kinetics and affinity constants.

FIG. 13: rPlg binding to cell receptors (HEK293 cells). rPLG binds to mammalian cell Plg receptor.

FIG. 14: rPlg (labelled with Alexa Fluor 790) accumulates at site of bone and muscular injury and reduces dystrophic calcification following injury. A. Top panel: rPlg accumulates at the bone fracture site. Alexa fluor labelled fibrin and rPlg was injected IP. Images show accumulation of fibrin and rPlg at the fracture site. Bottom panel: rPlg accumulates at muscle injury sites. Cardiotoxin was injected into the right leg to induce muscle injury, rPlg was given 1 mg/day IP. Images show accumulation of rPlg at the injured leg and kidneys but not the uninjured one.

B. Top panel: rPlg accumulation at muscle injury site. Cardiotoxin was injected into the leg to induce muscle injury. rPlg labelled with Alex Fluor dye was injected IP at 1 mg/day and images were recorded at days 1-7 post-injury. Bottom panel: rPlg accumulation at the injury site prevents muscle calcification in Plg+/− animals. Cardiotoxin was injected into the leg to induce muscle injury. rPlg was injected IP at 1 mg/day and images were recorded at day 7 post-injury. Muscle calcification is evident in Plg+/− but not in WT animals, and can be rescued by using rPlg or inhibition of alpha2-antiplasmin (α2AP) expression using an α2AP antisense oligonucleotide.

FIG. 15: Results of co-expression trials using α2-AP. Representative SDS-PAGE gel (A) and Western blot (B) showing the results of co-expression of plasminogen with either PAI-1 or α2-AP. Lanes 1-3: Plg/PAI-1, days 1, 3 and 5; Lanes 4-6: Plg/α2-AP, days 1, 3 and 5;

FIG. 16: Results of co-expression trials using PAI-2 and PAI-3. A. Representative Western blot showing the results of co-expression of plasminogen with PAI-1, PAI-2, or PAI-3. Lanes 1-3: Plg/PAI-1 stable, days 1, 3 and 5. Lanes 4-6: Plg/PAI-1, days 1, 3 and 5; Lanes 7-9 Plg/PAI-2, days 1, 3 and 5; and Lanes 10-12: Plg/PAI-3, days 1, 3 and 5. B and C. Plasminogen activity (RFU/s) for recoverable recombinant plasminogen obtained when co-expressed with PAI-1, PAI-2 or PAI-3. Note in 16B the lines with little to no RFU are Plg+PAI-2 and Plg+PAI-3, whereas the curve denoting significant RFU is Plg+PAI-1 stable and Plg+PAI-1.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

Expression of large amounts of proteins in a recombinant expression system is a convenient way to obtain protein for use in clinical applications. However, there have been significant difficulties in successfully producing intact plasminogen in various expression systems. This has previously been attributed to the ubiquitous presence of intracellular plasminogen activators in mammalian cells (which degrade the plasminogen or result in toxicity in the cell). Expression in non-mammalian systems has also been investigated, but has limited application in a clinical setting given the formation of inclusion bodies when expressed in bacterial systems, and limitations with appropriate protein folding and glycosylation in bacterial and non-mammalian eukaryotic systems. As a result of the inherent difficulties in obtaining sufficient amounts and plasminogen having the requisite purity and activity for use in a clinical setting, most plasminogen produced for use in clinical settings today is derived from fractionation of human plasma.

The present inventors have developed a new approach for producing plasminogen in a recombinant system. Surprisingly, the inventors have been able to utilize a mammalian expression system to produce significant quantities of recombinant plasminogen. Moreover, the inventors have demonstrated that the recombinant protein produced is biologically active and in fact has superior efficacy when compared to commercially available preparations of plasminogen, purified from plasma.

In addition, the method developed by the inventors is easy to use, whereby the recombinant plasminogen, which is free of pathogen contaminants, can be purified in an easy 3-step process. Thus, the inventors have developed a robust, simple method for obtaining functional, and pure plasminogen in sufficient quantities for use in a clinical setting.

The inventors have surprisingly found that co-expression of plasminogen with plasminogen activator inhibitor (PAI-1) enables large quantities of full-length, functional plasminogen to be produced recombinantly in mammalian cells. The yields obtained by the inventors provide for a significant improvement over prior art methods for recombinant expression of plasminogen which have involved co-expression of other components of the plasmin/fibrinolysis pathway.

### Plasminogen

Plasminogen is the inactive precursor form of plasmin, the principal fibrinolytic enzyme in mammals. Plasmin also plays an important role in cell migration, tissue remodeling, and bacterial invasion. Plasmin is a seine protease that preferentially cleaves Lys-Xaa and Arg—Xaa bonds with higher selectivity than trypsin. Plasminogen activators such as tissue plasminogen activator (tPA) or urokinase cleave human plasminogen molecule at the Arg₅₆₀-Val₅₆₁ bond to produce active plasmin. The two resulting chains of plasmin are held together by two interchain disulphide bridges. The light chain (25 kDa) carries the catalytic center (which comprises the catalytic triad) and shares sequence similarity with trypsin and other serine proteases. The heavy chain (60 kDa) consists of five highly similar triple-loop structures called kringles. Some of the kringles contain lysine binding sites that mediates the plasminogen/plasmin interaction with fibrin. Plasmin belongs to peptidase family Si.

The amino acid sequence of human Glu-Plg is provided in SEQ ID NO: 2 (see also SEQ ID NO:6). SEQ ID NO:16 shows the “mature” amino acid sequence, i.e. after cleavage of the signal peptide.

It will be understood that the present invention includes the recombinant production of plasminogen from human and non-human sources. Accordingly, the plasminogen produced according to the present methods may comprise of consist of the amino acid sequence any mammalian plasminogen or plasminogen variant. In any embodiment, the plasminogen is human plasminogen, non-human primate plasminogen, pig, mouse, rat, hamster, sheep, goat, horse, cow, cat, dog, or other mammalian plasminogen. Preferably, the plasminogen is human plasminogen.

Furthermore, the invention includes expression of functional variants of plasminogen including but not limited to those further described herein. More specifically, the present invention contemplates methods for the recombinant production of Glu-plasminogen (Glu-Plg), Lys-plasminogen (Lys-Pig), and mini-, midi- and micro-plasminogens.

Lys-plasminogen is an N-truncated form of Glu-Plg that is formed from the cleavage of Glu-plasminogen by plasmin. Lys-plasminogen exhibits higher affinity for fibrin compared to Glu-Plg and is better activated by uPA and tPA.

The amino acid sequence of human Lys-plasminogen is provided in SEQ ID NO: 9. SEQ ID NO:17 shows the “mature” amino acid sequence, i.e., after cleavage of the signal peptide.

Midi-plasminogen comprises kringle domains 4 and 5 and the light chain (serine protease domain) of plasminogen. It is formed by cleavage of kringle domains 1 to 3 from Glu-plasminogen.

The amino acid sequence of human midi-plasminogen is provided in SEQ ID NO:11. SEQ ID NO:18 shows the “mature” amino acid sequence, i.e., after cleavage of the signal peptide.

Mini-plasminogen (also known as 442Val-Plg or neoplasminogen) results from the action of elastase on Glu-plasminogen at residue 442 (located within Kringle domain 4). Thus mini-plasminogen comprises part of kringle domain 4, kringle domain 5 and the serine protease domain of plasminogen. The amino acid sequence of human mini-plasminogen is provided in SEQ ID NO:13. SEQ ID NO:19 shows the “mature” amino acid sequence, i.e., after cleavage of the signal peptide.

Micro-plasminogen consists of the proenzyme domain of plasminogen with a stretch of connecting peptide and a few residues of kringle 5 attached at its N-terminal end. It is produced by the action of plasmin on plasminogen. Thus, micro-plasmingogen (or micro-Pig) comprises the light chain of plasminogen (serine protease domain) and no kringle domains. (See, for example, Shi et al. (1980) J Biol. Chem. 263:17071-5). Like plasminogen, microplasminogen is activated by tPA and urokinase to form a proteolytically active molecule. Human microplasmin has a molecular weight of approximately 29 kDa and has a lower affinity for fibrin when compared with plasmin.

The amino acid sequence of human micro-plasminogen is provided in SEQ ID NO:15. SEQ ID NO:20 shows the “mature” amino acid sequence, i.e., after cleavage of the signal peptide.

Other variants: eg: variants of plasminogen which comprise modifications or mutations in the lysine binding sites found in the kringle domains. It will be appreciated that the methods of the invention lend themselves to expression of any of a number of Plasminogen variants, including but not limited to recombinant plasminogen having a modification at one or more sites.

PAI-1

Plasminogen activator inhibitor-1 (PAI-1) also known as endothelial plasminogen activator inhibitor or serpin E1 is a protein that in humans is encoded by the SERPINE1 gene. PAI-1 is a serine protease inhibitor (serpin) that functions as the principal inhibitor of tissue plasminogen activator (tPA) and urokinase (uPA), the activators of plasminogen. In vivo, PAI-1 is thus one or the key inhibitors of fibrinolysis.

Other plasminogen activator inhibitors include plasminogen activator inhibitor-2 (PAI-2), protein C inhibitor (PAI-3) and the protease nexin-1 (SERPINE2), which acts as an inhibitor of tPA and urokinase. The present inventors have found however, that the methods of the invention have particular utility when PAI-1 or a variant thereof, is co-expressed with plasminogen.

The amino acid sequence of human PAI-1, wherein the sequence is modified at Q197 and G355 to introduce cysteine residues, is provided in SEQ ID NO: 4.

Exemplary nucleic acid and amino acid sequences for PAI-2 and PAI-3 are provided in NCBI accession numbers NM_002575.3 and NM_000624.6, respectively.

It will be understood that the present invention also contemplates the use of “wild-type” PAI-1 (i.e., wherein the sequence is not modified at residues 197 or G355 as shown in SEQ ID NO: 4.

As used herein, the term “mutant” with respect to a mutant polypeptide or mutant polynucleotide is used interchangeably with “variant.” A variant with respect to a given reference sequence can include naturally occurring allelic variants. A “variant” includes any protein or amino acid sequence comprising at least one amino acid mutation with respect to wild-type. Mutations may include substitutions, insertions, and deletions. Preferably the variant retains the capacity to inhibit a plasminogen activator to the same exact as wildtype, or to a level of at least about 99%, 98%, 97%, 96%, 96%, 04%, 93%, 92%, 91%, 90%, 85%, or 80% of wildtype. For example a PAI-1 variant retains the capacity to inhibit a plasminogen activator to a level of at least about 99%, 98%, 97%, 96%, 96%, 04%, 93%, 92%, 91%, 90%, 85%, or 80% of wildtype PAI-1. The wildtype PAI-1 may be any described herein including SEQ ID NO: 4. In one embodiment, a variant of PAI-1 is not PAI-2 or PAI-3. Preferably, a PAI-1 variant has greater potency at inhibiting tPA and/or uPA compared to PAI-2, PAI-3, or PAI-2 and PAI-3.

### Nucleic Acids

An “isolated” nucleic acid molecule is a nucleic acid molecule that is identified and separated from at least one contaminant nucleic acid molecule with which it is ordinarily associated in the natural source. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in cells that ordinarily express, for example, plasminogen, where, for example, the nucleic acid molecule is in a chromosomal location different from that of natural cells.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides of the invention can be synthesised according to methods well known in the art, as described by way of example in Sambrook et al (1989, Molecular Cloning—a laboratory manual; Cold Spring Harbor Press).

The polynucleotide molecules of the present invention may be provided in the form of an expression cassette which includes control sequences operably linked to the inserted sequence, thus allowing for expression of the polypeptide of the invention in vivo in a targeted subject. These expression cassettes, in turn, are typically provided within vectors (e.g., plasmids or recombinant viral vectors) which are suitable for use as reagents for nucleic acid immunization. Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences.

Furthermore, it will be appreciated that the compositions and products of the invention may comprise a mixture of polypeptides and polynucleotides. Accordingly, the invention provides a composition or product as defined herein, wherein in place of any one of the polypeptide is a polynucleotide capable of expressing said polypeptide.

Expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art.

Thus, the methods of the present invention include delivering such a vector to a cell and allowing transcription from the vector to occur. Preferably, a polynucleotide of the invention or for use in the invention in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extra-chromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus. Thus, a peptide of the invention may be provided to an individual by expression from cells within the individual, and secretion from those cells.

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6: 47 (1988)), and established cell lines such as the COS-7, C127, 3T3, CHO, HeLa, and BHK cell lines, and the like. The skilled person is aware that the choice of expression host has ramifications for the type of polypeptide produced. For instance, the glycosylation of polypeptides produced in yeast or mammalian cells (e.g., COS-7 cells) will differ from that of polypeptides produced in bacterial cells, such as Escherichia coli.

### Polypeptides

“Isolated,” when used to describe the various polypeptides disclosed herein, means the polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes polypeptide in situ within recombinant cells, since at least one component of the polypeptide natural environment will not be present. Ordinarily, however, isolated polypeptide will be prepared by at least one purification step.

A “fragment” is a portion of a polypeptide of the present invention that retains substantially similar functional activity or substantially the same biological function or activity as the polypeptide, which can be determined using assays described herein.

“Percent (%) amino acid sequence identity” or “percent (%) identical” with respect to a polypeptide sequence, i.e. a polypeptide of the invention defined herein, is defined as the percentage of amino acid residues in a candidate sequence that are identical with the amino acid residues in the specific polypeptide of the invention, after aligning the sequences and introducing gaps, if necessary, to achieve the maximum percent sequence identity, and not considering any conservative substitutions as part of the sequence identity.

Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting examples of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, Calif., USA). When utilizing the ALIGN program for comparing amino acid sequences, a PAM 120 weight residue table, a gap length penalty of 12, and a gap penalty of 4 can be used.

The polypeptide desirably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo.

The polypeptide can be prepared by any of a number of conventional techniques. The polypeptide can be isolated or purified from a naturally occurring source or from a recombinant source. Recombinant production is preferred. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1982); Sambrook et al., Molecular Cloning A Laboratory Manual, 2nd ed. (Cold Spring Harbor Laboratory, 1989). The fragment can be transcribed and the polypeptide subsequently translated in vitro. Commercially available kits also can be employed (e.g., such as manufactured by Clontech, Palo Alto, Calif.; Amersham Pharmacia Biotech Inc., Piscataway, N.J.; InVitrogen, Carlsbad, Calif., and the like). The polymerase chain reaction optionally can be employed in the manipulation of nucleic acids.

The term “conservative substitution” as used herein, refers to the replacement of an amino acid present in the native sequence in the peptide with a naturally or non-naturally occurring amino acid or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

Conservative amino acid substitution tables providing functionally similar amino acids are well known to one of ordinary skill in the art. The following six groups are examples of amino acids that may be considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

As naturally occurring amino acids are typically grouped according to their properties, conservative substitutions by naturally occurring amino acids can be determined bearing in mind the fact that replacement of charged amino acids by sterically similar non-charged amino acids are considered as conservative substitutions. For producing conservative substitutions by non-naturally occurring amino acids it is also possible to use amino acid analogs (synthetic amino acids) well known in the art. A peptidomimetic of the naturally occurring amino acid is well documented in the literature known to the skilled person and non-natural or unnatural amino acids are described further below. When affecting conservative substitutions the substituting amino acid should have the same or a similar functional group in the side chain as the original amino acid.

The phrase “non-conservative substitution” or a “non-conservative residue” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH2)5-COOH]—CO— for aspartic acid. Non-conservative substitution includes any mutation that is not considered conservative.

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Alterations of the native amino acid sequence to produce mutant polypeptides, such as by insertion, deletion and/or substitution, can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used, such as disclosed in Walder et al., Gene 42: 133 (1986); Bauer et al., Gene 37: 73 (1985); Craik, Biotechniques, 12-19 (January 1995); and U.S. Pat. Nos. 4,518,584 and 4,737,462. A preferred means for introducing mutations is the QuikChange Site-Directed Mutagenesis Kit (Stratagene, LaJolla, Calif.).

The terms “N-terminal” and “C-terminal” are used herein to designate the relative position of any amino acid sequence or polypeptide domain or structure to which they are applied. The relative positioning will be apparent from the context. That is, an “N-terminal” feature will be located at least closer to the N-terminus of the polypeptide molecule than another feature discussed in the same context (the other feature possible referred to as “C-terminal” to the first feature). Similarly, the terms “5′-” and “3′-” can be used herein to designate relative positions of features of polynucleotides.

A recombinant polypeptide made in accordance with the methods of the present invention may also be modified by, conjugated or fused to another moiety to facilitate purification of the polypeptides, or for use in immunoassays using methods known in the art. For example, a polypeptide of the invention may be modified by glycosylation, acetylation, phosphorylation, amidation, derivatization by known protecting/blocking groups, proteolytic cleavage, etc.

Modifications contemplated herein include, but are not limited to, modification to side chains, incorporating of unnatural amino acids and/or their derivatives during polypeptide synthesis and the use of crosslinkers and other methods which impose conformational constraints on the polypeptides of the invention. Any modification, including post-translational modification, that reduces the capacity of the molecule to form a dimer is contemplated herein. An example includes modification incorporated by click chemistry as known in the art. Exemplary modifications include glycosylation.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α- | Mgabu | L-N-methylarginine | Nmarg |
| methylbutyrate | | | |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Deys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α- | Dmasn | α-methyl-α-napthylalanine | Manap |
| methylasparagine | | | |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α- | Dmmet | N-(2-carbamylethyl)glycine | Ngln |
| methylmethionine | | | |
| D-α-methylornithine | Dmorn | N-(carbamylmethyl)glycine | Nasn |
| D-α- | Dmphe | N-(2-carboxyethyl)glycine | Nglu |
| methylphenylalanine | | | |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α- | Dmtrp | N-cyclohexylglycine | Nchex |
| methyltryptophan | | | |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N- | Dnmasn | N-cycloundecylglycine | Ncund |
| methylasparagine | | | |
| D-N-methylaspartate | Dnmasp | N-(2,2- | Nbhm |
| | | diphenylethyl)glycine | |
| D-N-methylcysteine | Dnmcys | N-(3,3- | Nbhe |
| | | diphenylpropyl)glycine | |
| D-N- | Dnmgln | N-(3- | Narg |
| methylglutamine | | guanidinopropyl)glycine | |
| D-N- | Dnmglu | N-(1-hydroxyethyl)glycine | Nthr |
| methylglutamate | | | |
| D-N-methylhistidine | Dnmhis | N-(hydroxyethyl))glycine | Nser |
| D-N- | Dnmile | N-(imidazolylethyl))glycine | Nhis |
| methylisoleucine | | | |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclo- | Nmchexa | D-N-methylmethionine | Dnmmet |
| hexylalanine | | | |
| D-N-methylornithine | Dnmorn | N- | Nmcpen |
| | | methylcyclopentylalanine | |
| N-methylglycine | Nala | D-N-methylphenylalanine | Dnmphe |
| N-methylamino- | Nmaib | D-N-methylproline | Dnmpro |
| isobutyrate | | | |
| N-(1-methyl- | Nile | D-N-methylserine | Dnmser |
| propyl)glycine | | | |
| N-(2-methyl- | Nleu | D-N-methylthreonine | Dnmthr |
| propyl)glycine | | | |
| D-N- | Dnmtrp | N-(1-methylethyl)glycine | Nval |
| methyltryptophan | | | |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p- | Nhtyr |
| | | hydroxyphenyl)glycine | |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α- | Mhphe |
| | | methylhomophenylalanine | |
| L-α-methylisoleucine | Mile | N-(2- | Nmet |
| | | methylthioethyl)glycine | |
| L-α-methylleucine | Mleu | L-α-methyllysine | Mlys |
| L-α- | Mmet | L-α-methylnorleucine | Mnle |
| methylmethionine | | | |
| L-α-methylnorvaline | Mnva | L-α-methylornithine | Morn |
| L-α- | Mphe | L-α-methylproline | Mpro |
| methylphenylalanine | | | |
| L-α-methylserine | Mser | L-α-methylthreonine | Mthr |
| L-α- | Mtrp | L-α-methyltyrosine | Mtyr |
| methyltryptophan | | | |
| L-α-methylvaline | Mval | L-N- | Nmhphe |
| | | methylhomophenylalanine | |
| N-(N-(2,2- | Nnbhm | N-(N-(3,3-diphenylpropyl) | Nnbhe |
| diphenylethyl) | | carbamylmethyl)glycine | |
| carbamylmethyl) | | | |
| glycine | | | |
| 1-carboxy-1- | | | |
| (2,2-diphenyl-Nmbc | | | |
| ethylamino) | | | |
| cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The polypeptides referred to herein as having an N-terminal domain “homologous to a kringle domain of native human plasminogen” exhibit structural and functional characteristics similar to native kringle domains of plasminogen. Further, the polypeptides referred to herein as having an N-terminal domain “homologous to kringle 1” exhibit characteristics similar to native kringle 1, at least to the extent that the polypeptides can have a higher affinity for o-aminocarboxylic acids (and functional homologs such as trans-4-aminomethylcyclohexane-15 carboxylic acid, a cyclic acid) than kringle 5. See, e.g., Chang, Y., et al., Biochemistry 37:3258-3271 (1998), incorporated herein by reference, for conditions and protocols for comparison of binding of isolated kringle domain polypeptides to aminopentanoic acid (5-APnA); 6-aminohexanoic acid (6-AHxA), also known as epsilon-aminocaprioic acid (EACA); 7-aminoheptanoic acid (7-AHpA); and trans-4aminomethylcyclohexane-1-carboxylic acid (t-AMCHA). References to kringle domains “homologous to kringle 4” are defined similarly, as noted above regarding the phrase “homologous to kringle 1.” That is, they exhibit functional characteristics similar to kringle 4 of native human plasminogen as discussed above. These polypeptides also bind immobilized lysine as described above.

The polypeptides made according to the methods of the present invention bind immobilized lysine. As used herein, the phrase “binding immobilized lysine” means that the polypeptides so characterized are retarded in their progress relative to proteins that do not bind lysine, when subjected to column chromatography using lysine-SEPHAROSE as the chromatographic media. Typically, the polypeptides of the invention can be eluted from such chromatographic media (lysine affinity resins) using solutions containing the specific ligand, e.g., EACA, as eluants.

Cell Culture

Persons skilled in the art will be familiar with standard methods for transfecting host cells, such as mammalian cells, with a nucleic acid vector and culturing the host cell in suitable conditions for expressing genes encoded by the vector. Representative methods for transfection and culturing of mammalian cells to produce recombinant protein are described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989).

Means for introducing the isolated nucleic acid, vector or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

The host cells used in accordance with the present invention may be cultured in a variety of media, depending on the cell type used. Commercially available media such as Ham's Fl0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

Moreover, the skilled person will be familiar with methods for purifying expressed recombinant protein from cell culture media, including using size exclusion and affinity chromatography methods, and combinations thereof.

Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., lysine affinity column), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Assaying the Activity of the Recombinant Plasminogen

The recombinant plasminogen produced according to the present invention (or the recombinant plasmin derived therefrom) can be assessed for biological activity using standard methods known in the art and as described later herein in the Examples.

For example, the recombinant plasminogen can be converted to plasmin via cleavage with tPA or uPA using standard techniques. Cleavage of recombinant plasminogen with tPA yields heavy and light chains comprising residues Glu₁-Arg₅₆₁ and Val₅₆₂-Asn₇₉₁, respectively (for an example of the method for converting plasminogen to plasmin, see Mutch and Booth, Chapter 20 in Hemostasis and Thrombosis: Basic Principles and Clinical Practice by Victor J. Marder, William C. Aird, Joel S. Bennett, Sam Schulman, and II Gilbert C. White, incorporated herein by reference).

Further, the ability of the recombinant plasminogen to bind to physiological binding targets or ligands can be assessed using conventional techniques. For example, binding to recombinant plasminogen (or plasmin derived therefrom) can be assessed in relation to binding to alpha 2-antiplasmin (α2-AP) and streptokinase. (Methods for assessing binding to α2-AP and to streptokinase are described in, for example, Horvath et al., (2011) Methods in Enzymology, 501: 223-235 and in Zhang et al., (2012) Journal of Biological Chemistry, 287: 42093-42103, respectively, the contents of which are hereby incorporated by reference.

The binding of the recombinant proteins produced according to the present invention to cell surface receptors such as the mammalian plasminogen receptor can be determined, for example as described in Example 4.

Finally, the therapeutic efficacy of the recombinant proteins produced according to the present invention can be used according to standard techniques, including those techniques utilised for assessment of the quality of plasminogen and plasmin isolated from human and non-human plasma.

Compositions

The recombinant plasminogen (or plasmin derived therefrom) can be provided in a pharmaceutically acceptable composition for administration to an individual in need thereof. For example the recombinant proteins made in accordance with the present invention find utility in the treatment of wounds (such as dermal wounds, including abrasions and burns, bone wounds, including bone fractures muscle injury), in providing replacement plasminogen in the context of traumatic injury, in plasminogen replacement therapy where there is a congenital deficiency, treatment of heterotopic ossification and dystrophic calcification.

In some examples, the recombinant plasminogen (or plasmin derived therefrom) as described herein can be administered parenterally, topically, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing a recombinant plasminogen into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

Pharmaceutical compositions of this disclosure are particularly useful for parenteral administration, such as intravenous administration or administration into a body cavity or lumen of an organ or joint. The compositions for administration will commonly comprise a solution of plasminogen dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of plasminogen of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, a recombinant plasminogen made in accordance with the present disclosure will be administered in a manner compatible with the dosage formulation and in such amount as is therapeutically/prophylactically effective.

### EXAMPLES

### Example 1: Transient Expression of Recombinant Plasminogen

Materials

Expi293 expression medium (CAT #A1435101), Erlenmeyer flask, Phosphate buffered saline (1×), Glucose (300 mg/mL), polyethylenimine PEI (1 mg/mL), Lupin (125 g/L), Glutamax (100X), pcDNA3.1 Plg (0.5 μg/mL cell culture), pcDNA3.1 PAI-1 (0.5 μg/mL cell culture)

Method

Day 1: Dilute cells prior to transfection.

Day 2: Add DNA diluted in PBS and PEI to cells. Incubate cells at 37° C. 5% CO₂ at 110-140 rpm.

Days 3-7: Adjust lupin and glucose levels in culture media.

Day 8: Harvest medium by centrifuging culture at 2000×g at 4° C. for 15 minutes.

### Example 2: Stable Expression of Recombinant Plasminogen

Materials

Expi293 expression medium (CAT #A1435101) supplemented with 4 g/L lupin and 125 mg/L geneticin (G418), Erlenmeyer flask, Glucose (300 mg/mL), Glutamax (100×).

Method
Expi293 cells were transfected with pcDNA 3.1 Plg RES PAI-1.
Cells were then passaged in the presence of selection antibiotic geneticin
Geneticin-resistant clones were separated into 96-well plate (1 colony per well)
Presence of secreted Plg was tested using streptokinase.

Typical yields from transient expression are 30-50 mg/L of cell culture. Typical yields rom stably transfected Expi293 cells (suitable for large-scale expression) are in the order of 80-100 mg/L cell culture.

### Example 3: Purification of Recombinant Plasminogen

For every 100 mL of clarified supernatant, 20 mL of 0.5 M NaH₂PO₄, 5 g of glycerol and 1 Roche protease inhibitor tablet (comprising aprotinin, bestatin, calpain inhibitor I and II, chymostatin, E-64, Leupeptin, pefabloc SC/PMSF, pepstatin, TLCK-HCl, Trypsin inhibitor, Antipain dihydrochloride, phopsphoramidonare) added and mixed.

1. Lysine affinity column

Buffer A: 100 mM Na₂HPO₄, pH 8.0, 5% glycerol, 0.02% azide

Buffer B: 100 mM Na₂HPO₄ pH 8.0, 25 mM EACA (epsilon aminocaproic acid), 5% glycerol

CV=column volume

a) Use 20 mL Lysine Hyper D resin per 100 mL of culture supernatant.

b) In a gravity-flow column, wash the resin with 2 CV of MQ H₂O and equilibrate with 2 CV of Buffer A

c) Add equilibrated lysine resin to clarified media (from step 1) and batch-bind for 1 hour at 4° C.

d) Allow media to flow-through and collect flow-through

e) Wash resin with 2 CV buffer A.

f) Elute with buffer B- typically with half resin volume at a time. (For example, 10 mL fractions for 20 mL resin). Use Bradford's reagent to determine elution endpoint

g) Run fractions on a 10% SDS-PAGE.

h) Pool fractions for next purification step.

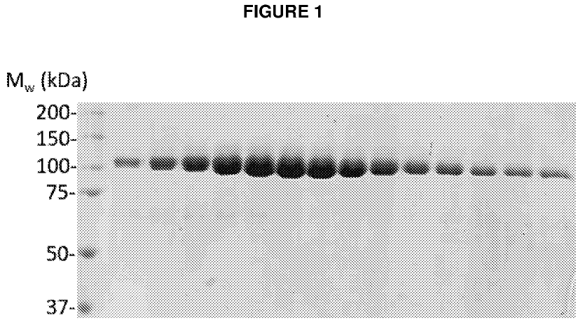

FIG. 1 shows a representative Coomassie-stained 10% SDS-PAGE gel image of fractions eluted from lysine affinity column. Bands at around 100 kDa represent eluted rPlg, following transient expression.

2. Hi Trap Q FF

Buffer A: 50 mM Tris pH 9.0, 5 mM EACA, 10% glycerol, 30 mM NaCl, 0.02% azide

Buffer B: 50 mM Tris pH 9.0, 5 mM EACA, 10% glycerol, 1M NaCl, 0.02% azide

a) Concentrate sample to 5 ml (50K MWCO), keep pre-column sample and dilute to 50 ml using buffer A

b) Pre-equilibrate HiTrap Q with 5CV of MQ H₂O and 5CV buffer A

c) Load sample at 1 ml/min and collect flow-through.

d) Wash with 5 CV of buffer A

e) Elution gradient:
0 to 25% B in 20 CV (100 ml)
20% to 100% B in 0 CV
100% for 2CV

Plg is typically eluted as the dominant peak at around 10% B. Run gel to determine purity and pool fractions.

Dialyze pooled fractions overnight at 4° C. in 25 mM Tris pH 7.4, 150 mM NaCl, 5% glycerol. Repeat dialysis for another two hours. This is to ensure the removal of EACA.

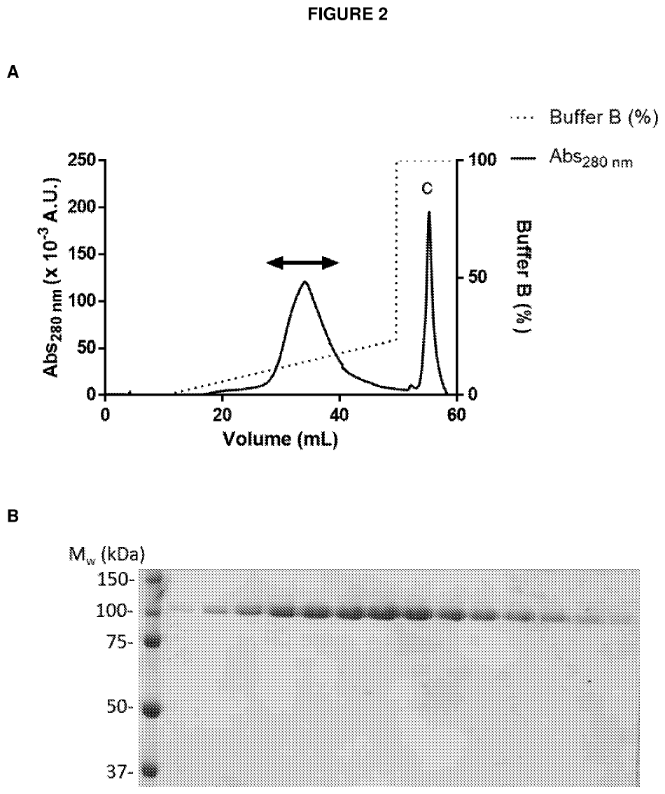

FIG. 2 shows the representative results of anion exchange chromatography of rPlg.

3. Gel filtration S200 16/60

Buffer: 25 mM Tris pH 7.4, 150 mM NaCl, 5% glycerol, 1 mM sodium EDTA, 0.02% azide, lx Roche protease inhibitor cocktail. (Tris can be substituted with Hepes or Na₂HPO₄)

a) Concentrate to at least 5 ml and gel fil on Superdex 200 16/60.

b) Elution volume is around 73 ml.

c) Run gel and pool the relevant fractions.

In the presence of 5% glycerol, plasminogen can be concentrated up to 15 mg/ml in a 50K MWCO concentrator.

Plasminogen can be stored frozen after snap-freezing in liquid N₂.

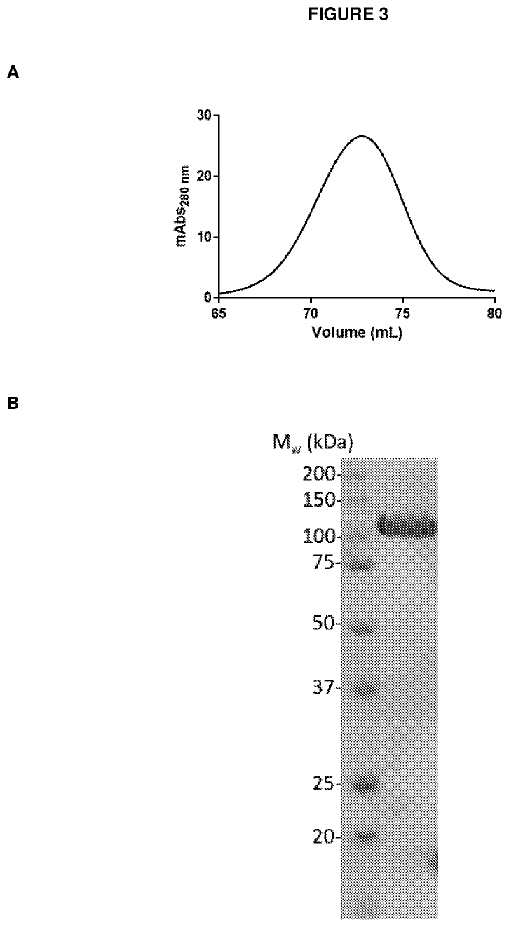

FIG. 3 shows the representative results of size-exclusion chromatography of rPlg.

Mini-plg and micro-plg were also successfully expressed and purified using the methods described herein and shown to be active (data not shown).

### Example 4: Qualitative Assessment of Recombinant Plasminogen

1. rPlg can be activated into plasmin (rPlm)

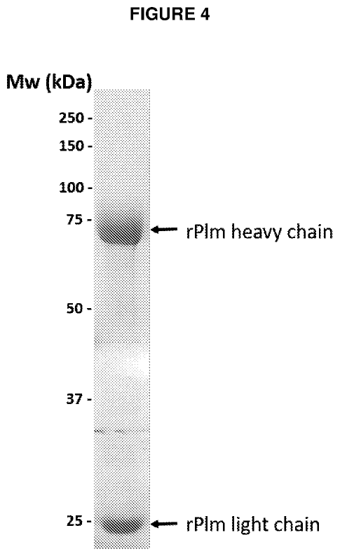

FIG. 4 shows a Coomassie-stained 12% SDS-PAGE showing tPA-cleaved recombinant plasmin (rPlm) resulting from the cleavage of rPlg by tPA. Under reducing conditions, rPlm is separated into heavy and light chains, which comprise of residues Glu₁-Arg₅₆₁ and Val₅₆₂-Asn₇₉₁, respectively.

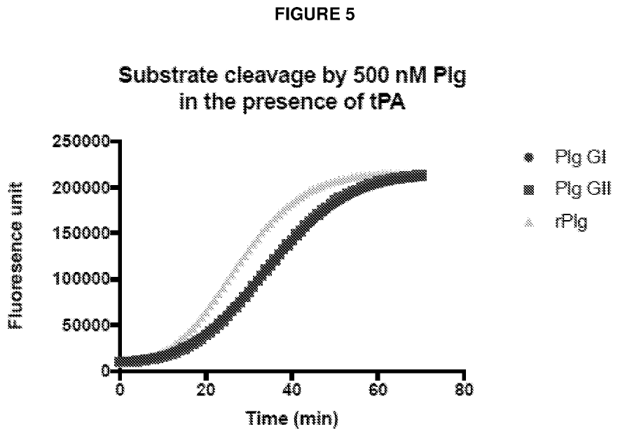

FIG. 5 shows a progress curve showing tPA activation of 500 nM rPlg was measured by the hydrolysis of plasmin fluorogenic substrate H-Ala-Phe-Lys-AMC.

2. rPlg is more similar to native Plg GI

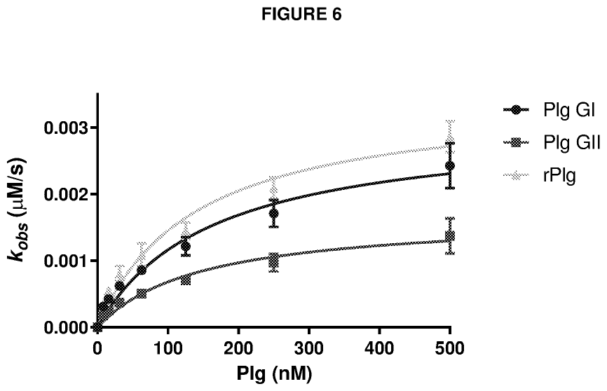

FIG. 6 shows tPA activation of native Plg glycoform 1 (GLI), Plg glycoform II (GII) and rPlg. rPlg was obtained according to the methods described herein. Native Plg Glycoforms I and II were obtained in-house and purified from human plasma using standard techniques.

The results indicate that rPlg is the most readily activatable from the three forms tested (having the lowest Kₘ and highest Vₘₐₓ).

| | Plg Gl | Plg Gll | rPlg |
|---|---|---|---|
| Kₘ (nM) | 163 ± 30 | 146 ± 30 | 139 ± 22 |
| Vₘₐₓ (nM/s) | 3.06 ± 0.24 | 1.67 ± 0.14 | 3.47 ± 0.22 |

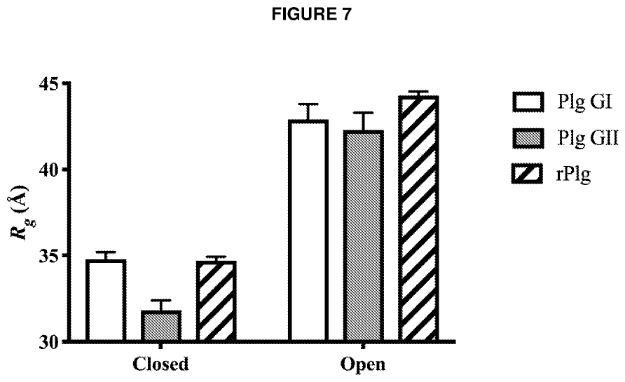

FIG. 7 shows that the open confirmation of Plg is induced by the presence of 20 mM EACA. In the closed form, glycoform I and rPlg have similar and higher radius of giration (R_{g}) than glycoform II. (Radius of gyration of a body about an axis of rotation is defined as the radial distance of a point from the axis of rotation at which, if the whole mass of the body is assumed to be concentrated, its moment of inertia about the given axis would be the same as with its actual distribution of mass. X-ray scattering can also be used to measure the over dimensions of a rotating macromolecule in solution. Plg can assume both closed and open conformations. The dimension of the open forms is very similar. In the closed form, a small R_{g} indicates a small and tightly packed molecule. This measure is used as an indication of how stable/well-packed the closed form is.)

| | Plg Gl | Plg Gll | rPlg |
|---|---|---|---|
| Closed R_{g} (Å) | 34.8 ± 0.4 | 31.8 ± 0.6 | 34.7 ± 0.2 |
| Open R_{g} (Å) | 42.9 ± 0.9 | 42.3 ± 1.0 | 44.3 ± 0.2 |

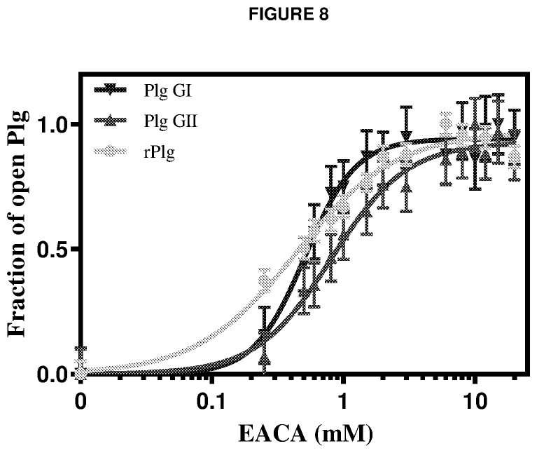

A SAXS titration study (FIG. 8) measured the conformational change of Plg in response to EACA. The titration curves are similar between glycoforms I and II and rPlg. rPlg adopts the open form most readily. Kₒₚₑₙ is the EACA concentration at which 50% of Plg is in the open conformation.

| | Plg Gl | Plg Gll | rPlg |
|---|---|---|---|
| Kₒₚₑₙ (mM) | 0.51 ± 0.02 | 0.81 ± 0.06 | 0.44 ± 0.04 |

3. rPlm binds to the Plm-specific inhibitor alpha 2-antiplasmin (α2-AP)

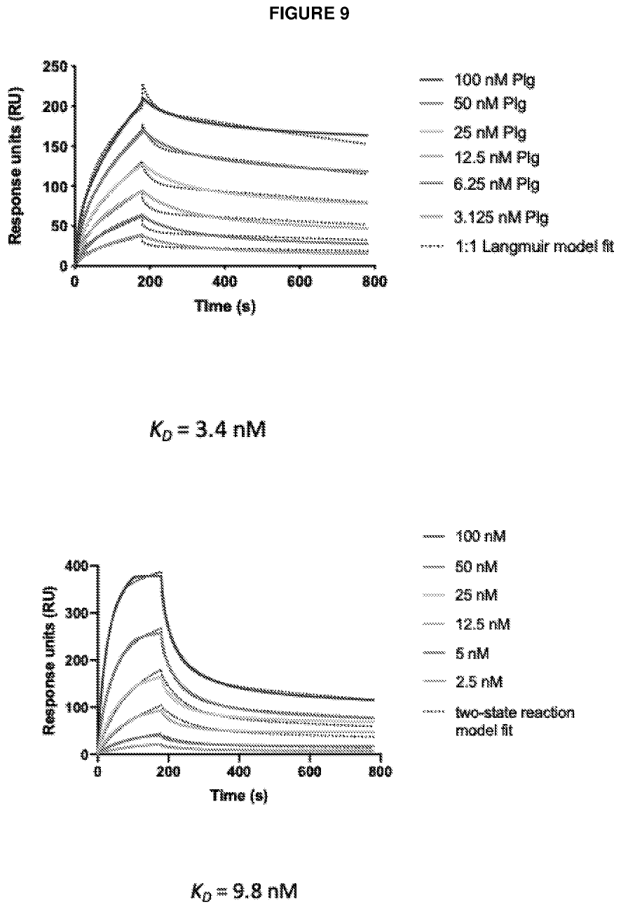

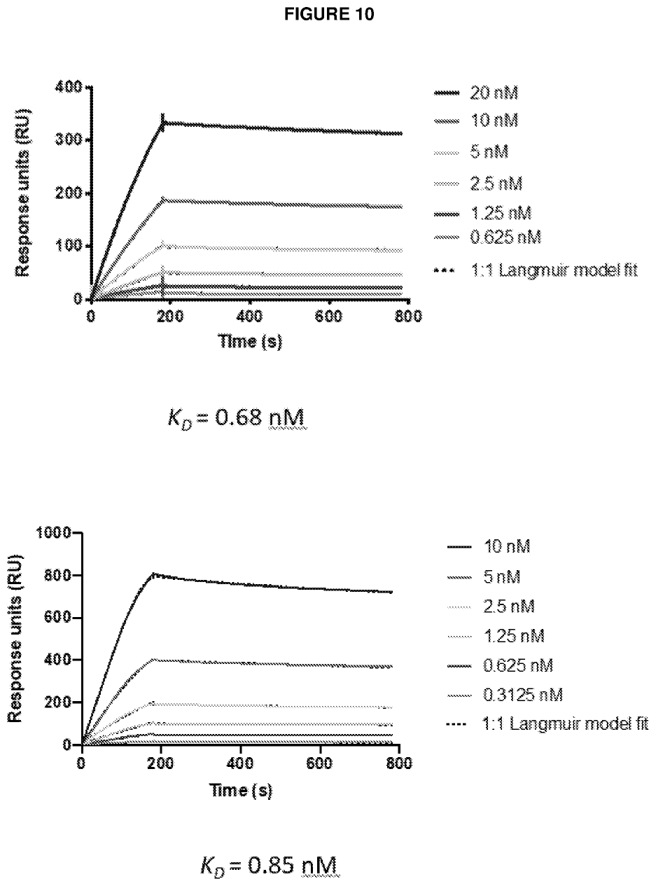

Recombinant α2-AP was immobilized on a Ni2+−NTA chip and the binding of native or recombinant Plg and Plm were monitored in real time. FIGS. 9 and 10 show sensorgrams demonstrating binding of rPlg and rPlm as well as native Plg and Plm to alpha 2-antiplasmin (α2-AP).

rPlg and rPlm were obtained by the methods described herein. Native Plg was purchased from Merck (purified from human plasma) and native Plm was purchased from Haematologic Technologies.

4. rPlg and rPlm are bound by Streptokinase from Streptococcus pyrogenes

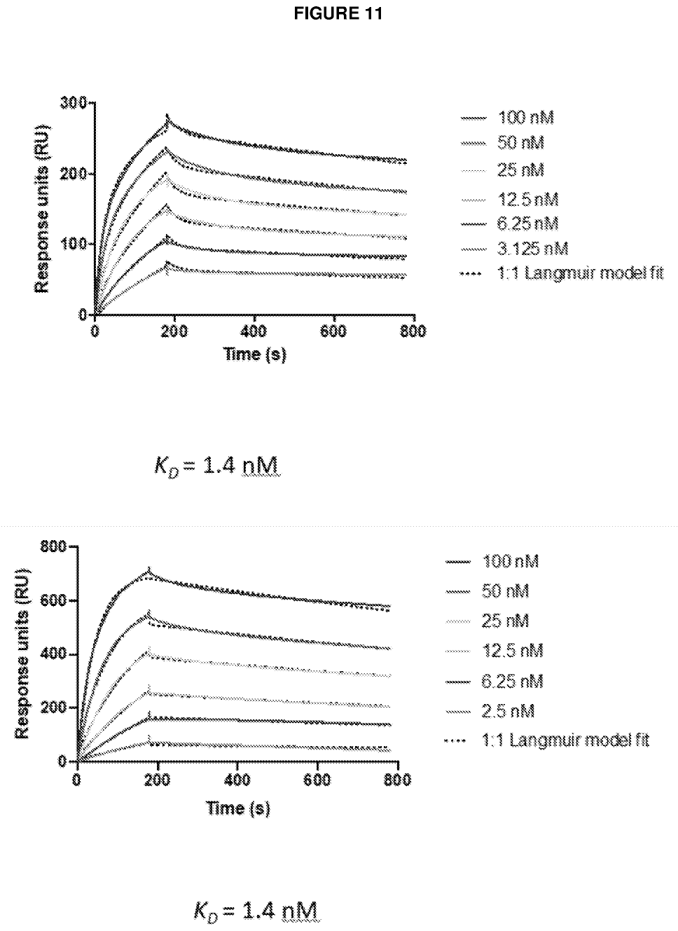

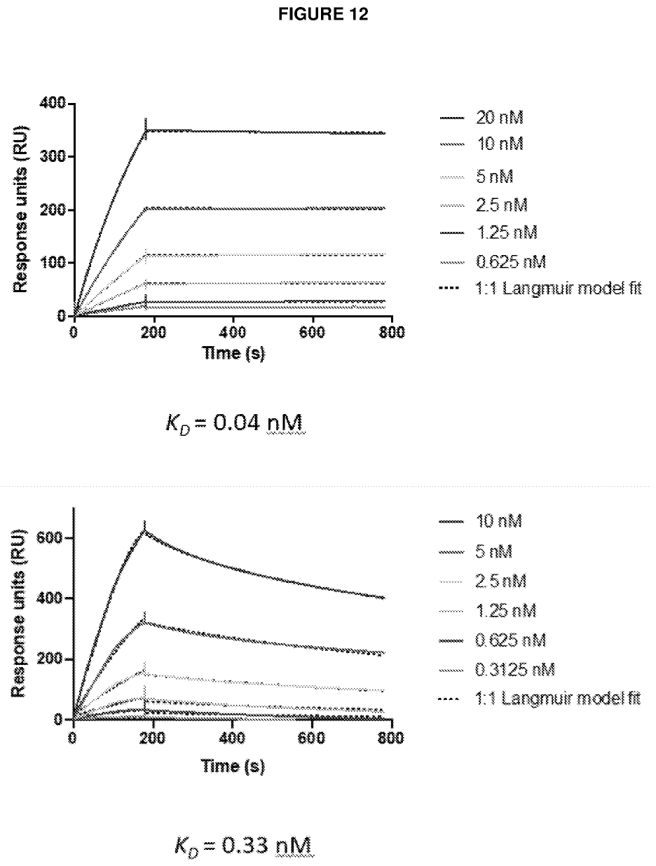

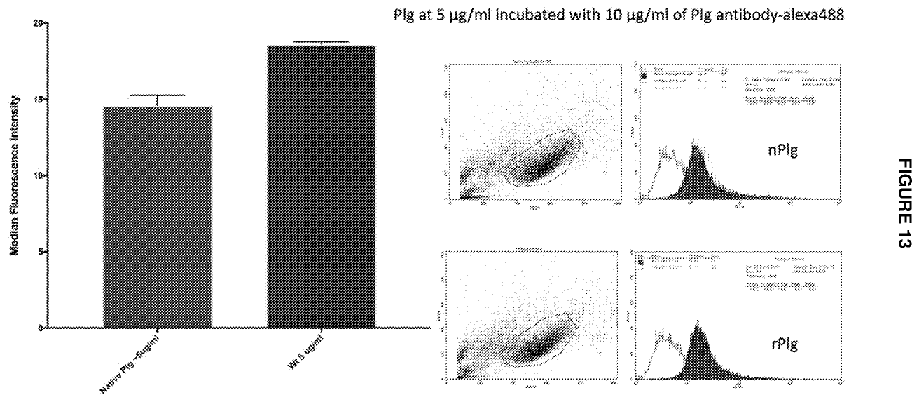

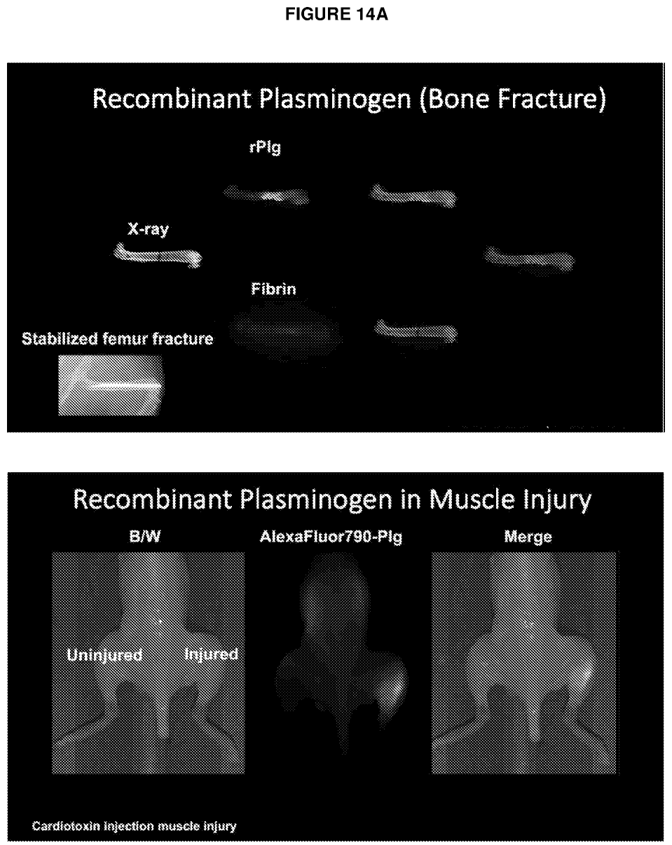

Recombinant SK was immobilized on a Ni2+−NTA chip and the binding of native or recombinant Plg and Plm were monitored in real time. rPlg and rPlm were obtained by the methods described herein. Native Plg was purchased from Merck (purified from human plasma) and native Plm was purchased from Haematologic Technologies. The results are shown in FIGS. 11 and 12.

All experiments were fitted with a 1:1 Langmuir binding model (except for α2-AP/native Plg, where a two-state reaction model was used to describe the conformational change of Plg glycoform II upon binding) using the Biacore T200 evaluation software (Biacore AB). Kinetics (kₐ and k_{d}) and affinity constants (K_{D}) are tabulated below:

| | kₐ₁(M⁻¹.s⁻¹) | k_{d1}(s⁻¹) | kₐ₂(s⁻¹) | k_{d2}(s⁻¹) | K_{D}(M) |
|---|---|---|---|---|---|
| α2-AP/native Plg | 1.1 × 10⁶ | 0.065 | 0.0033 | 6.8 × 10⁻⁴ | 9.8 × 10⁻⁹ |

| | kₐ(M⁻¹.s⁻¹) | k_{d}(s⁻¹) | K_{D}(M) |
|---|---|---|---|
| α2-AP/recombinant Plg | 1.5 × 10⁵ | 5.0 × 10⁻⁴ | 3.4 × 10⁻⁹ |
| α2-AP/native Plm | 5.1 × 10⁶ | 4.3 × 10⁻³ | 8.5 × 10⁻¹⁰ |
| α2-AP/recombinant Plm | 1.6 × 10⁵ | 1.1 × 10⁻⁴ | 6.8 × 10⁻¹⁰ |
| SK/native Plg | 2.3 × 10⁵ | 3.2 × 10⁻⁴ | 1.4 × 10⁻⁹ |
| SK/recombinant Plg | 2.7 × 10⁵ | 3.6 × 10⁻⁴ | 1.4 × 10⁻⁹ |
| SK/native Plm | 7.1 × 10⁶ | 2.3 × 10⁻³ | 3.3 × 10⁻¹⁰ |
| SK/recombinant Plm | 4.1 × 10⁵ | 1.6 × 10⁻⁵ | 4.0 × 10⁻¹¹ |

The above results demonstrate that recombinant plasminogen produced according to the methods of the present invention (or plasmin derived therefrom), binds to physiologically relevant binding partners with similar or greater affinity than commercially available preparations of native plasminogen/plasmin purified from plasma.

5. rPlg binds to mammalian Plg receptor

Briefly, HEK293 cells were resuspended PBS-EDTA+2% FCS. 5×10⁵ cells per sample were then incubated for 30 minutes with nPlg or rPlg at 5 ug/mL and Alexa-488 labelled Plg antibody at 10 ug/mL. Median Fluorescence Intensity of at least 10,000 events was measured on a FACSCalibur (BD Biosciences) flow cytometer on the FL1 channel (488 nm laser excitation source). FIG. 13 shows binding of rPlg to plasminogen receptor in HEK293 cells.

6. rPlg accumulates at the site of bone and muscular injury and reduces dystrophic calcification following injury

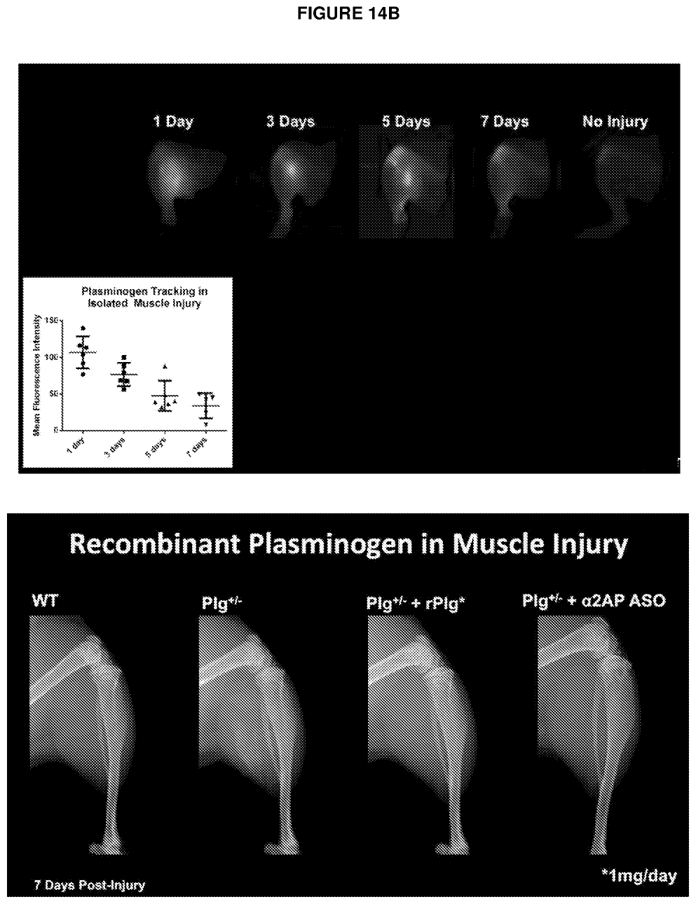

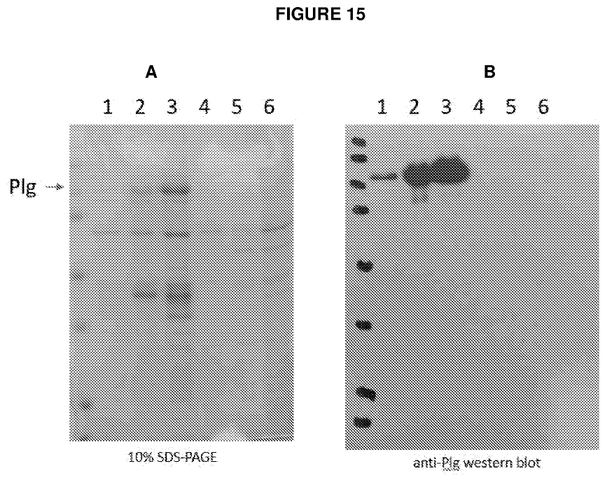

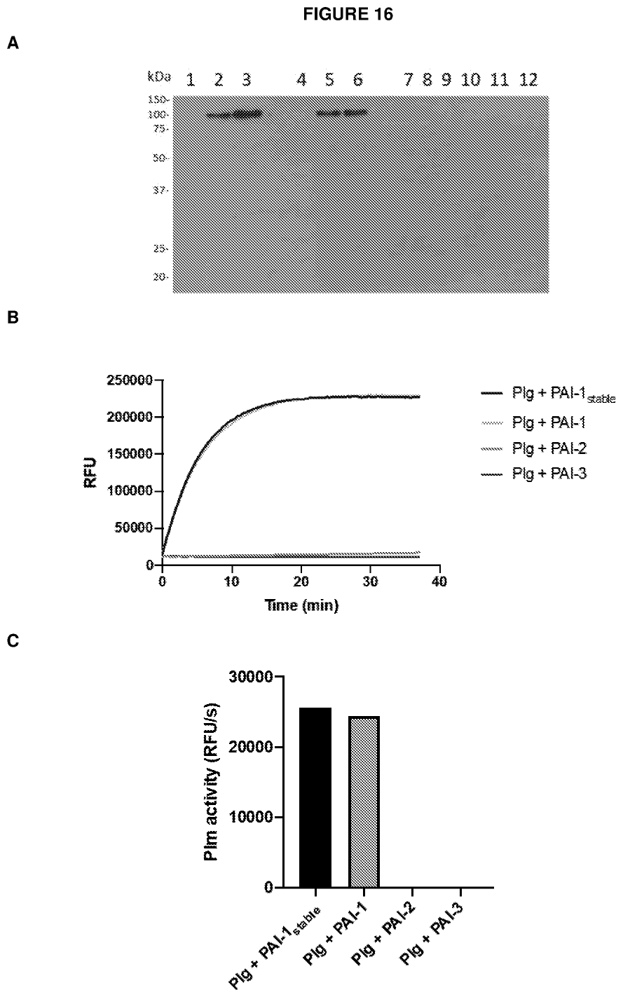

FIG. 14 shows the results following injection of rPlg subsequent to bone and muscle injury. A. Top panel shows that rPlg accumulates at the fracture site in bone. Alexa fluor labelled fibrin and rPlg were injected IP. Images show accumulation of fibrin and rPlg at the fracture site. Bottom pane shows that rPlg accumulates at the site of muscle injury. Cardiotoxin was injected into the right leg to induce muscle injury and rPlg was given 1 mg/day IP. Images show accumulation of rPlg at the injured leg and kidneys but not in uninjured leg/kidney.

B. Top panel also shows rPlg accumulation at the site of muscle injury. Cardiotoxin was injected into the leg to induce muscle injury. rPlg labelled with Alex Fluor dye was injected IP at 1 mg/day and images were recorded at days 1-7 post-injury. Bottom panel: rPlg accumulation at the site of injury prevents muscle calcification in Plg+/− animal. Cardiotoxin was injected into the leg to induce muscle injury. rPlg was injected IP at 1 mg/day and images were recorded at day 7 post-injury. Muscle calcification is evident in Plg+/− but not in WT animal, and can be rescued by using rPlg or inhibition of alpha2-antiplasmin (α2AP) expression using an α2AP antisense oligonucleotide.

### Example 5: Co-Expression of Recombinant Plasminogen with Other Inhibitors

Expi293 cells were co-transfected with constructs encoding:
recombinant Plg/α2-AP;
recombinant Plg/PAI-1_{stable};
recombinant Plg/PAI-1;
recombinant Plg/PAI-2;
recombinant Plg/PAI-3.

All constructs were pre-mixed at a 1:1 (w/w) ratio prior to transfection.

Expression level of Plg at 1, 3 and 5 days post-transfection was determined by western blot using anti-Plg antibody.

The results, shown in FIGS. 15 and 16 demonstrate that yield of recombinant Plg was significantly higher when co-expressed with stably-transfected PAI-1 or transiently transfected PAI-1, compared to when co-expressed with any of α2-AP, PAI-2 or PAI-3.

Conclusion: PAI-1 is important for the expression of recombinant Plg. The results from the experiments comparing expression of Plg when co-expressed with α-AP indicate that the inhibition of Plm activation is more important than the inhibition of Plm activity, in order to maximise protein yield.

Sequence information
Exemplary nucleic acid sequence of human
plasminogen
(SEQ ID NO: 1)
ATGGAACACAAAGAAGTGGTGTTGCTCCTGCTGCT
GTTCCTGAAGTCCGGCCAGGGCGAGCCCCTGGACG
ATTACGTGAACACCCAGGGCGCCAGCCTGTTCAGC
GTGACCAAGAAACAGCTGGGAGCCGGCAGCATCGA
GGAATGCGCCGCCAAGTGCGAAGAGGACGAGGAAT
TCACCTGTCGGGCCTTCCAGTACCACAGCAAAGAA
CAGCAGTGCGTGATCATGGCCGAGAACAGAAAGAG
CAGCATCATCATCAGAATGCGGGACGTGGTGCTGT
TCGAGAAGAAGGTGTACCTGAGCGAGTGCAAGACC
GGCAACGGCAAGAACTACCGGGGCACCATGAGCAA
GACCAAGAACGGCATCACCTGTCAGAAGTGGTCCA
GCACCAGCCCCCACCGGCCTAGATTTTCTCCAGCC
ACCCACCCTAGCGAGGGCCTGGAAGAGAACTACTG
CCGGAACCCCGACAACGACCCTCAGGGCCCTTGGT
GCTACACCACCGACCCCGAGAAGAGATACGACTAC
TGCGACATCCTGGAATGTGAAGAGGAATGCATGCA
CTGCAGCGGCGAGAACTACGACGGCAAGATCTCCA
AGACCATGAGCGGCCTGGAATGCCAGGCTTGGGAC
AGCCAGTCTCCTCACGCCCACGGCTACATCCCCAG
CAAGTTCCCCAACAAGAACCTGAAGAAGAATTACT
GCAGAAACCCTGACCGCGAGCTGCGGCCCTGGTGT
TTTACCACCGATCCTAACAAGAGATGGGAGCTGTG
CGATATCCCCCGGTGCACCACACCTCCACCTAGCA
GCGGCCCTACCTACCAGTGTCTGAAGGGCACCGGC
GAGAATTACAGGGGCAACGTGGCCGTGACCGTGTC
CGGCCATACCTGCCAGCATTGGAGCGCCCAGACCC
CCCACACCCACAACAGAACCCCCGAGAACTTCCCC
TGCAAGAATCTGGACGAGAATTATTGTCGCAACCC
CGATGGCAAGAGGGCCCCCTGGTGTCACACCACCA
ACAGCCAGGTGCGCTGGGAGTACTGCAAGATCCCC
AGCTGCGATAGCAGCCCCGTGTCCACAGAACAGCT
GGCCCCTACAGCCCCTCCTGAGCTGACACCTGTGG
TGCAGGATTGCTACCACGGCGACGGCCAGAGCTAC
AGAGGCACCAGCAGCACCACCACAACCGGCAAGAA
GTGCCAGAGCTGGTCCTCCATGACCCCTCACCGGC
ACCAGAAAACCCCTGAGAATTACCCCAACGCCGGC
CTGACCATGAACTACTGTAGAAATCCCGACGCCGA
CAAGGGACCCTGGTGCTTCACAACAGACCCTTCCG
TCAGATGGGAATACTGTAATCTGAAGAAGTGCAGC
GGCACCGAGGCCAGCGTGGTGGCTCCTCCACCAGT
GGTGCTGCTGCCCGATGTGGAAACCCCCTCCGAAG
AGGACTGTATGTTCGGCAATGGCAAGGGCTATAGA
GGCAAGCGGGCCACCACCGTGACCGGCACACCTTG
TCAGGATTGGGCCGCTCAGGAACCCCACAGACACA
GCATCTTCACCCCAGAGACAAACCCTCGGGCCGGA
CTGGAAAAAAACTATTGTCGGAATCCTGACGGCGA
CGTGGGAGGACCTTGGTGTTATACAACAAACCCAC
GGAAGCTGTACGATTACTGTGACGTGCCCCAGTGT
GCCGCCCCTAGCTTCGATTGTGGCAAGCCCCAGGT
GGAACCCAAGAAATGCCCCGGCAGAGTCGTGGGCG
GATGTGTGGCCCATCCTCACTCTTGGCCTTGGCAG
GTGTCCCTGCGGACCAGATTCGGCATGCACTTTTG
CGGCGGCACCCTGATCAGCCCCGAGTGGGTGCTGA
CAGCCGCCCACTGTCTGGAAAAGTCCCCCAGACCC
AGCAGCTACAAAGTGATCCTGGGAGCCCACCAGGA
AGTGAACCTGGAACCTCACGTGCAGGAAATCGAGG
TGTCCAGACTGTTCCTGGAACCCACCCGGAAGGAT
ATCGCCCTGCTGAAGCTGAGCAGCCCTGCCGTGAT
CACCGACAAAGTGATTCCCGCCTGCCTGCCCAGCC
CCAACTATGTGGTGGCCGACAGAACCGAGTGCTTC
ATCACCGGCTGGGGCGAGACACAGGGCACATTTGG
AGCCGGCCTGCTGAAAGAGGCCCAGCTGCCTGTGA
TCGAGAACAAAGTGTGCAACCGCTACGAGTTCCTG
AACGGCAGAGTGCAGAGCACCGAGCTGTGTGCCGG
ACATCTGGCTGGCGGCACAGATAGCTGTCAGGGCG
ATTCTGGCGGCCCTCTCGTGTGCTTCGAGAAGGAC
AAGTACATCCTGCAGGGCGTGACCAGCTGGGGCCT
GGGATGTGCCAGACCTAACAAGCCCGGCGTGTACG
TGCGCGTGTCCAGATTTGTGACCTGGATCGAGGGC
GTGATGCGGAACAACTGA
Exemplary amino acid sequence of human
plasminogen
Signal peptide underlined.
(SEQ ID NO: 2)
MEHKEVVLLLLLFLKSGQG EPLDDYVNTQGASLFS
VTKKQLGAGSIEECAAKCEEDEEFTCRAFQYHSKE
QQCVIMAENRKSSIIIRMRDVVLFEKKVYLSECKT
GNGKNYRGTMSKTKNGITCQKWSSTSPHRPRFSPA
THPSEGLEENYCRNPDNDFOGPWCYTTDPEKRYDY
CDILECEEECMHCSGENYDGKISKTMSGLECQAWD
SQSPHAHGYIPSKFPNKNLKKNYCRNPDRELRPWC
FTTDPNKRWELCDIPRCTTPPPSSGPTYQCLKGTG
ENYRGNVAVTVSGHTCQHWSAQTPHTHNRTPENFP
CKNLDENYCRNPDGKRAPWCHTTNSQVRWEYCKIP
SCDSSPVSTEQLAPTAPPELTPVVQDCYHGDGQSY
RGTSSTTTTGKKCQSWSSMTPHRHQKTPENYPNAG
LTMNYCRNPDADKGPWCFTTDPSVRWEYCNLKKCS
GTEASVVAPPPVVLLPDVETPSEEDCMFGNGKGYR
GKRATTVTGTPCQDWAAQEPHRHSIFTPETNPRAG
LEKNYCRNPDGDVGGPWCYTTNPRKLYDYCDVPQC
AAPSFDCGKPQVEPKKCPGRVVGGCVAHPHSWPWQ
VSLRTRFGMHFCGGTLISPEWVLTAAHCLEKSPRP
SSYKVILGAHQEVNLEPHVQEIEVSRLFLEPTRKD
IALLKLSSPAVITDKVIPACLPSPNYVVADRTECF
ITGWGETQGTFGAGLLKEAQLPVIENKVCNRYEFL
NGRVQSTELCAGHLAGGTDSCQGDSGGPLVCFEKD
KYILQGVTSWGLGCARPNKPGVYVRVSRFVTWIEG
VMRNN
Exemplary nucleic acid sequence of
recombinant PAI-1.
Includes mutation: 0197C and G355C
(underlined in the sequence below)
to improve serpin stability (per
Chorostowska-Wynimko J et al.
(2003) Molecular Cancer Therapeutics.
2003; 2(1):19-28.
doi: 10.1186/1476-4598-2-19)
(SEQ ID NO: 3)
ATGCAGATGTCTCCCGCCCTGACCTGCCTGGTGCT
GGGCCTGGCCCTGGTGTTCGGAGAGGGCTCTGCCG
TGCACCACCCACCTAGCTACGTGGCACACCTGGCC
TCCGACTTCGGCGTGAGGGTGTTTCAGCAGGTGGC
CCAGGCCAGCAAGGATCGCAACGTGGTGTTCAGCC
CTTATGGCGTGGCCTCCGTGCTGGCCATGCTCCAG
CTGACCACAGGAGGAGAGACCCAGCAGCAGATCCA
GGCAGCTATGGGCTTCAAGATCGACGATAAGGGAA
TGGCACCCGCCCTGAGGCACCTGTACAAGGAGCTG
ATGGGCCCTTGGAATAAGGACGAGATCAGCACCAC
AGATGCCATCTTTGTGCAGCGCGACCTGAAGCTGG
TGCAGGGCTTCATGCCACACTTCTTTCGGCTGTTC
CGGAGCACCGTGAAGCAGGTGGACTTCAGCGAGGT
GGAGAGGGCCCGCTTTATCATCAACGATTGGGTGA
AGACCCACACAAAGGGCATGATCAGCAATCTGCTG
GGCAAGGGAGCAGTGGATCAGCTGACCAGGCTGGT
GCTGGTGAACGCCCTGTACTTCAATGGCTGCTGGA
AGACCCCATTTCCCGACAGCTCCACACACCGGAGA
CTGTTCCACAAGTCCGATGGCTCTACAGTGAGCGT
GCCTATGATGGCCCAGACCAACAAGTTCAATTATA
CAGAGTTTACCACACCTGACGGCCACTACTATGAC
ATCCTGGAGCTGCCATACCACGGCGACACCCTGAG
CATGTTTATCGCCGCCCCTTATGAGAAGGAGGTGC
CACTGTCCGCCCTGACAAACATCCTGTCCGCCCAG
CTGATCTCTCACTGGAAGGGCAATATGACCAGGCT
GCCAAGGCTGCTGGTGCTGCCTAAGTTCTCCCTGG
AGACAGAGGTGGACCTGCGGAAGCCTCTGGAGAAC
CTGGGCATGACCGATATGTTCAGACAGTTTCAGGC
CGACTTTACATCTCTGAGCGATCAGGAGCCACTGC
ACGTGGCACAGGCCCTCCAGAAGGTGAAGATCGAG
GTGAACGAGTCCTGTACCGTGGCCTCTAGCTCCAC
AGCCGTGATCGTGTCTGCCAGGATGGCCCCAGAGG
AGATCATCATGGATCGGCCCTTCCTGTTTGTGGTG
AGACACAATCCAACCGGCACAGTGCTGTTCATGGG
CCAGGTCATGGAGCCCTGA
Amino acid sequence of recombinant
PAI-1, Q197C, G355C
(SEQ ID NO: 4)
MQMSPALTCLVLGLALVFGEGSAVHHPPSYVAHLA
SDFGVRVFQQVAQASKDRNVVFSPYGVASVLAMLQ
LTTGGETQQQIQAAMGFKIDDKGMAPALRHLYKEL
MGPWNKDEISTTDAIFVQRDLKLVQGFMPHFFRLF
RSTVKQVDFSEVERARFIINDWVKTHTKGMISNLL
GKGAVDQLTRLVLVNALYFNGCWKTPFPDSSTHRR
LFHKSDGSTVSVPMMAQTNKFNYTEFTTPDGHYYD
ILELPYHGDTLSMFIAAPYEKEVPLSALTNILSAQ
LISHWKGNMTRLPRLLVLPKFSLETEVDLRKPLEN
LGMTDMFRQFQADFTSLSDQEPLHVAQALQKVKIE
VNESCTVASSSTAVIVSARMAPEEIIMDRPFLFVV
RHNPTGTVLFMGQVMEP
Nucleic acid sequence for hPlg-IRES2-
PAI-1 expression cassette (construct
for stable expression of plasminogen
and PAI-1)
(SEQ ID NO: 5)
ATGGAACACAAAGAAGTGGTGTTGCTCCTGCTGCT
GTTCCTGAAGTCCGGCCAGGGCGAGCCCCTGGACG
ATTACGTGAACACCCAGGGCGCCAGCCTGTTCAGC
GTGACCAAGAAACAGCTGGGAGCCGGCAGCATCGA
GGAATGCGCCGCCAAGTGCGAAGAGGACGAGGAAT
TCACCTGTCGGGCCTTCCAGTACCACAGCAAAGAA
CAGCAGTGCGTGATCATGGCCGAGAACAGAAAGAG
CAGCATCATCATCAGAATGCGGGACGTGGTGCTGT
TCGAGAAGAAGGTGTACCTGAGCGAGTGCAAGACC
GGCAACGGCAAGAACTACCGGGGCACCATGAGCAA
GACCAAGAACGGCATCACCTGTCAGAAGTGGTCCA
GCACCAGCCCCCACCGGCCTAGATTTTCTCCAGCC
ACCCACCCTAGCGAGGGCCTGGAAGAGAACTACTG
CCGGAACCCCGACAACGACCCTCAGGGCCCTTGGT
GCTACACCACCGACCCCGAGAAGAGATACGACTAC
TGCGACATCCTGGAATGTGAAGAGGAATGCATGCA
CTGCAGCGGCGAGAACTACGACGGCAAGATCTCCA
AGACCATGAGCGGCCTGGAATGCCAGGCTTGGGAC
AGCCAGTCTCCTCACGCCCACGGCTACATCCCCAG
CAAGTTCCCCAACAAGAACCTGAAGAAGAATTACT
GCAGAAACCCTGACCGCGAGCTGCGGCCCTGGTGT
TTTACCACCGATCCTAACAAGAGATGGGAGCTGTG
CGATATCCCCCGGTGCACCACACCTCCACCTAGCA
GCGGCCCTACCTACCAGTGTCTGAAGGGCACCGGC
GAGAATTACAGGGGCAACGTGGCCGTGACCGTGTC
CGGCCATACCTGCCAGCATTGGAGCGCCCAGACCC
CCCACACCCACAACAGAACCCCCGAGAACTTCCCC
TGCAAGAATCTGGACGAGAATTATTGTCGCAACCC
CGATGGCAAGAGGGCCCCCTGGTGTCACACCACCA
ACAGCCAGGTGCGCTGGGAGTACTGCAAGATCCCC
AGCTGCGATAGCAGCCCCGTGTCCACAGAACAGCT
GGCCCCTACAGCCCCTCCTGAGCTGACACCTGTGG
TGCAGGATTGCTACCACGGCGACGGCCAGAGCTAC
AGAGGCACCAGCAGCACCACCACAACCGGCAAGAA
GTGCCAGAGCTGGTCCTCCATGACCCCTCACCGGC
ACCAGAAAACCCCTGAGAATTACCCCAACGCCGGC
CTGACCATGAACTACTGTAGAAATCCCGACGCCGA
CAAGGGACCCTGGTGCTTCACAACAGACCCTTCCG
TCAGATGGGAATACTGTAATCTGAAGAAGTGCAGC
GGCACCGAGGCCAGCGTGGTGGCTCCTCCACCAGT
GGTGCTGCTGCCCGATGTGGAAACCCCCTCCGAAG
AGGACTGTATGTTCGGCAATGGCAAGGGCTATAGA
GGCAAGCGGGCCACCACCGTGACCGGCACACCTTG
TCAGGATTGGGCCGCTCAGGAACCCCACAGACACA
GCATCTTCACCCCAGAGACAAACCCTCGGGCCGGA
CTGGAAAAAAACTATTGTCGGAATCCTGACGGCGA
CGTGGGAGGACCTTGGTGTTATACAACAAACCCAC
GGAAGCTGTACGATTACTGTGACGTGCCCCAGTGT
GCCGCCCCTAGCTTCGATTGTGGCAAGCCCCAGGT
GGAACCCAAGAAATGCCCCGGCAGAGTCGTGGGCG
GATGTGTGGCCCATCCTCACTCTTGGCCTTGGCAG
GTGTCCCTGCGGACCAGATTCGGCATGCACTTTTG
CGGCGGCACCCTGATCAGCCCCGAGTGGGTGCTGA
CAGCCGCCCACTGTCTGGAAAAGTCCCCCAGACCC
AGCAGCTACAAAGTGATCCTGGGAGCCCACCAGGA
AGTGAACCTGGAACCTCACGTGCAGGAAATCGAGG
TGTCCAGACTGTTCCTGGAACCCACCCGGAAGGAT
ATCGCCCTGCTGAAGCTGAGCAGCCCTGCCGTGAT
CACCGACAAAGTGATTCCCGCCTGCCTGCCCAGCC
CCAACTATGTGGTGGCCGACAGAACCGAGTGCTTC
ATCACCGGCTGGGGCGAGACACAGGGCACATTTGG
AGCCGGCCTGCTGAAAGAGGCCCAGCTGCCTGTGA
TCGAGAACAAAGTGTGCAACCGCTACGAGTTCCTG
AACGGCAGAGTGCAGAGCACCGAGCTGTGTGCCGG
ACATCTGGCTGGCGGCACAGATAGCTGTCAGGGCG
ATTCTGGCGGCCCTCTCGTGTGCTTCGAGAAGGAC
AAGTACATCCTGCAGGGCGTGACCAGCTGGGGCCT
GGGATGTGCCAGACCTAACAAGCCCGGCGTGTACG
TGCGCGTGTCCAGATTTGTGACCTGGATCGAGGGC
GTGATGCGGAACAACTGAAAGCTTGGTACCGAGCT
CGGATCCCCCCTCTCCCTCCCCCCCCCCTAACGTT
ACTGGCCGAAGCCGCTTGGAATAAGGCCGGTGTGC
GTTTGTCTATATGTTATTTTCCACCATATTGCCGT
CTTTTGGCAATGTGAGGGCCCGGAAACCTGGCCCT
GTCTTCTTGACGAGCATTCCTAGGGGTCTTTCCCC
TCTCGCCAAAGGAATGCAAGGTCTGTTGAATGTCG
TGAAGGAAGCAGTTCCTCTGGAAGCTTCTTGAAGA
CAAACAACGTCTGTAGCGACCCTTTGCAGGCAGCG
GAACCCCCCACCTGGCGACAGGTGCCTCTGCGGCC
AAAAGCCACGTGTATAAGATACACCTGCAAAGGCG
GCACAACCCCAGTGCCACGTTGTGAGTTGGATAGT
TGTGGAAAGAGTCAAATGGCTCTCCTCAAGCGTAT
TCAACAAGGGGCTGAAGGATGCCCAGAAGGTACCC
CATTGTATGGGATCTGATCTGGGGCCTCGGTACAC
ATGCTTTACATGTGTTTAGTCGAGGTTAAAAAAAC
GTCTAGGCCCCCCGAACCACGGGGACGTGGTTTTC
CTTTGAAAAACACGATGATAATATGCAGATGTCTC
CCGCCCTGACCTGCCTGGTGCTGGGCCTGGCCCTG
GTGTTCGGAGAGGGCTCTGCCGTGCACCACCCACC
TAGCTACGTGGCACACCTGGCCTCCGACTTCGGCG
TGAGGGTGTTTCAGCAGGTGGCCCAGGCCAGCAAG
GATCGCAACGTGGTGTTCAGCCCTTATGGCGTGGC
CTCCGTGCTGGCCATGCTCCAGCTGACCACAGGAG
GAGAGACCCAGCAGCAGATCCAGGCAGCTATGGGC
TTCAAGATCGACGATAAGGGAATGGCACCCGCCCT
GAGGCACCTGTACAAGGAGCTGATGGGCCCTTGGA
ATAAGGACGAGATCAGCACCACAGATGCCATCTTT
GTGCAGCGCGACCTGAAGCTGGTGCAGGGCTTCAT
GCCACACTTCTTTCGGCTGTTCCGGAGCACCGTGA
AGCAGGTGGACTTCAGCGAGGTGGAGAGGGCCCGC
TTTATCATCAACGATTGGGTGAAGACCCACACAAA
GGGCATGATCAGCAATCTGCTGGGCAAGGGAGCAG
TGGATCAGCTGACCAGGCTGGTGCTGGTGAACGCC
CTGTACTTCAATGGCTGCTGGAAGACCCCATTTCC
CGACAGCTCCACACACCGGAGACTGTTCCACAAGT
CCGATGGCTCTACAGTGAGCGTGCCTATGATGGCC
CAGACCAACAAGTTCAATTATACAGAGTTTACCAC
ACCTGACGGCCACTACTATGACATCCTGGAGCTGC
CATACCACGGCGACACCCTGAGCATGTTTATCGCC
GCCCCTTATGAGAAGGAGGTGCCACTGTCCGCCCT
GACAAACATCCTGTCCGCCCAGCTGATCTCTCACT
GGAAGGGCAATATGACCAGGCTGCCAAGGCTGCTG
GTGCTGCCTAAGTTCTCCCTGGAGACAGAGGTGGA
CCTGCGGAAGCCTCTGGAGAACCTGGGCATGACCG
ATATGTTCAGACAGTTTCAGGCCGACTTTACATCT
CTGAGCGATCAGGAGCCACTGCACGTGGCACAGGC
CCTCCAGAAGGTGAAGATCGAGGTGAACGAGTCCT
GTACCGTGGCCTCTAGCTCCACAGCCGTGATCGTG
TCTGCCAGGATGGCCCCAGAGGAGATCATCATGGA
TCGGCCCTTCCTGTTTGTGGTGAGACACAATCCAA
CCGGCACAGTGCTGTTCATGGGCCAGGTCATGGAG
CCCTGA
Exemplary nucleic acid sequence of human
dlu- Plg
(SEQ ID NO: 6)
ATGGAACACAAAGAAGTGGTGTTGCTCCTGCTGCT
GTTCCTGAAGTCCGGCCAGGGCGAGCCCCTGGACG
ATTACGTGAACACCCAGGGCGCCAGCCTGTTCAGC
GTGACCAAGAAACAGCTGGGAGCCGGCAGCATCGA
GGAATGCGCCGCCAAGTGCGAAGAGGACGAGGAAT
TCACCTGTCGGGCCTTCCAGTACCACAGCAAAGAA
CAGCAGTGCGTGATCATGGCCGAGAACAGAAAGAG
CAGCATCATCATCAGAATGCGGGACGTGGTGCTGT
TCGAGAAGAAGGTGTACCTGAGCGAGTGCAAGACC
GGCAACGGCAAGAACTACCGGGGCACCATGAGCAA
GACCAAGAACGGCATCACCTGTCAGAAGTGGTCCA
GCACCAGCCCCCACCGGCCTAGATTTTCTCCAGCC
ACCCACCCTAGCGAGGGCCTGGAAGAGAACTACTG
CCGGAACCCCGACAACGACCCTCAGGGCCCTTGGT
GCTACACCACCGACCCCGAGAAGAGATACGACTAC
TGCGACATCCTGGAATGTGAAGAGGAATGCATGCA
CTGCAGCGGCGAGAACTACGACGGCAAGATCTCCA
AGACCATGAGCGGCCTGGAATGCCAGGCTTGGGAC
AGCCAGTCTCCTCACGCCCACGGCTACATCCCCAG
CAAGTTCCCCAACAAGAACCTGAAGAAGAATTACT
GCAGAAACCCTGACCGCGAGCTGCGGCCCTGGTGT
TTTACCACCGATCCTAACAAGAGATGGGAGCTGTG
CGATATCCCCCGGTGCACCACACCTCCACCTAGCA
GCGGCCCTACCTACCAGTGTCTGAAGGGCACCGGC
GAGAATTACAGGGGCAACGTGGCCGTGACCGTGTC
CGGCCATACCTGCCAGCATTGGAGCGCCCAGACCC
CCCACACCCACAACAGAACCCCCGAGAACTTCCCC
TGCAAGAATCTGGACGAGAATTATTGTCGCAACCC
CGATGGCAAGAGGGCCCCCTGGTGTCACACCACCA
ACAGCCAGGTGCGCTGGGAGTACTGCAAGATCCCC
AGCTGCGATAGCAGCCCCGTGTCCACAGAACAGCT
GGCCCCTACAGCCCCTCCTGAGCTGACACCTGTGG
TGCAGGATTGCTACCACGGCGACGGCCAGAGCTAC
AGAGGCACCAGCAGCACCACCACAACCGGCAAGAA
GTGCCAGAGCTGGTCCTCCATGACCCCTCACCGGC
ACCAGAAAACCCCTGAGAATTACCCCAACGCCGGC
CTGACCATGAACTACTGTAGAAATCCCGACGCCGA
CAAGGGACCCTGGTGCTTCACAACAGACCCTTCCG
TCAGATGGGAATACTGTAATCTGAAGAAGTGCAGC
GGCACCGAGGCCAGCGTGGTGGCTCCTCCACCAGT
GGTGCTGCTGCCCGATGTGGAAACCCCCTCCGAAG
AGGACTGTATGTTCGGCAATGGCAAGGGCTATAGA
GGCAAGCGGGCCACCACCGTGACCGGCACACCTTG
TCAGGATTGGGCCGCTCAGGAACCCCACAGACACA
GCATCTTCACCCCAGAGACAAACCCTCGGGCCGGA
CTGGAAAAAAACTATTGTCGGAATCCTGACGGCGA
CGTGGGAGGACCTTGGTGTTATACAACAAACCCAC
GGAAGCTGTACGATTACTGTGACGTGCCCCAGTGT
GCCGCCCCTAGCTTCGATTGTGGCAAGCCCCAGGT
GGAACCCAAGAAATGCCCCGGCAGAGTCGTGGGCG
GATGTGTGGCCCATCCTCACTCTTGGCCTTGGCAG
GTGTCCCTGCGGACCAGATTCGGCATGCACTTTTG
CGGCGGCACCCTGATCAGCCCCGAGTGGGTGCTGA
CAGCCGCCCACTGTCTGGAAAAGTCCCCCAGACCC
AGCAGCTACAAAGTGATCCTGGGAGCCCACCAGGA
AGTGAACCTGGAACCTCACGTGCAGGAAATCGAGG
TGTCCAGACTGTTCCTGGAACCCACCCGGAAGGAT
ATCGCCCTGCTGAAGCTGAGCAGCCCTGCCGTGAT
CACCGACAAAGTGATTCCCGCCTGCCTGCCCAGCC
CCAACTATGTGGTGGCCGACAGAACCGAGTGCTTC
ATCACCGGCTGGGGCGAGACACAGGGCACATTTGG
AGCCGGCCTGCTGAAAGAGGCCCAGCTGCCTGTGA
TCGAGAACAAAGTGTGCAACCGCTACGAGTTCCTG
AACGGCAGAGTGCAGAGCACCGAGCTGTGTGCCGG
ACATCTGGCTGGCGGCACAGATAGCTGTCAGGGCG
ATTCTGGCGGCCCTCTCGTGTGCTTCGAGAAGGAC
AAGTACATCCTGCAGGGCGTGACCAGCTGGGGCCT
GGGATGTGCCAGACCTAACAAGCCCGGCGTGTACG
TGCGCGTGTCCAGATTTGTGACCTGGATCGAGGGC
GTGATGCGGAACAACTGA
Exemplary amino acid sequence of human
glu-Plg
Signal peptide underlined
(SEQ ID NO: 7)
MEHKEVVLLLLLFLKSGQGEPLDDYVNTQGASLFS
VTKKQLGAGSIEECAAKCEEDEEFTCRAFQYHSKE
QQCVIMAENRKSSIIIRMRDVVLFEKKVYLSECKT
GNGKNYRGTMSKTKNGITCQKWSSTSPHRPRFSPA
THPSEGLEENYCRNPDNDPQGPWCYTTDPEKRYDY
CDILECEEECMHCSGENYDGKISKTMSGLECQAWD
SQSPHAHGYIPSKFPNKNLKKNYCRNPDRELRPWC
FTTDPNKRWELCDIPRCTTPPPSSGPTYQCLKGTG
ENYRGNVAVTVSGHTCQHWSAQTPHTHNRTPENFP
CKNLDENYCRNPDGKRAPWCHTTNSQVRWEYCKIP
SCDSSPVSTEQLAPTAPPELTPVVQDCYHGDGQSY
RGTSSTTTTGKKCQSWSSMTPHRHQKTPENYPNAG
LTMNYCRNPDADKGPWCFTTDPSVRWEYCNLKKCS
GTEASVVAPPPVVLLPDVETPSEEDCMFGNGKGYR
GKRATTVTGTPCQDWAAQEPHRHSIFTPETNPRAG
LEKNYCRNPDGDVGGPWCYTTNPRKLYDYCDVPQC
AAPSFDCGKPQVEPKKCPGRVVGGCVAHPHSWPWQ
VSLRTRFGMHFCGGTLISPEWVLTAAHCLEKSPRP
SSYKVILGAHQEVNLEPHVQEIEVSRLFLEPTRKD
IALLKLSSPAVITDKVIPACLPSPNYVVADRTECF
ITGWGETQGTFGAGLLKEAQLPVIENKVCNRYEFL
NGRVQSTELCAGHLAGGTDSCQGDSGGPLVCFEKD
KYILQGVTSWGLGCARPNKPGVYVRVSRFVTWIEG
VMRNN
Exemplary nucleic acid sequence of
human Lys-Plg
(SEQ ID NO: 8)
atggaacacaaagaagtggtgttgctcctgctgct
gttcctgaagtccggccagggcaaggtgtacctga
gcgagtgcaagaccggcaacggcaagaactaccgg
ggcaccatgagcaagaccaagaacggcatcacctg
tcagaagtggtccagcaccagcccccaccggccta
gattttctccagccacccaccctagcgagggcctg
gaagagaactactgccggaaccccgacaacgaccc
tcagggcccttggtgctacaccaccgaccccgaga
agagatacgactactgcgacatcctggaatgtgaa
gaggaatgcatgcactgcagcggcgagaactacga
cggcaagatctccaagaccatgagcggcctggaat
gccaggcttgggacagccagtctcctcacgcccac
ggctacatccccagcaagttccccaacaagaacct
gaagaagaattactgcagaaaccctgaccgcgagc
tgcggccctggtgttttaccaccgatcctaacaag
agatgggagctgtgcgatatcccccggtgcaccac
acctccacctagcagcggccctacctaccagtgtc
tgaagggcaccggcgagaattacaggggcaacgtg
gccgtgaccgtgtccggccatacctgccagcattg
gagcgcccagaccccccacacccacaacagaaccc
ccgagaacttcccctgcaagaatctggacgagaat
tattgtcgcaaccccgatggcaagagggccccctg
gtgtcacaccaccaacagccaggtgcgctgggagt
actgcaagatccccagctgcgatagcagccccgtg
tccacagaacagctggcccctacagcccctcctga
gctgacacctgtggtgcaggattgctaccacggcg
acggccagagctacagaggcaccagcagcaccacc
acaaccggcaagaagtgccagagctggtcctccat
gacccctcaccggcaccagaaaacccctgagaatt
accccaacgccggcctgaccatgaactactgtaga
aatcccgacgccgacaagggaccctggtgcttcac
aacagacccttccgtcagatgggaatactgtaatc
tgaagaagtgcagcggcaccgaggccagcgtggtg
gctcctccaccagtggtgctgctgcccgatgtgga
aaccccctccgaagaggactgtatgttcggcaatg
gcaagggctatagaggcaagcgggccaccaccgtg
accggcacaccttgtcaggattgggccgctcagga
accccacagacacagcatcttcaccccagagacaa
accctcgggccggactggaaaaaaactattgtcgg
aatcctgacggcgacgtgggaggaccttggtgtta
tacaacaaacccacggaagctgtacgattactgtg
acgtgccccagtgtgccgcccctagcttcgattgt
ggcaagccccaggtggaacccaagaaatgccccgg
cagagtcgtgggcggatgtgtggcccatcctcact
cttggccttggcaggtgtccctgcggaccagattc
ggcatgcacttttgcggcggcaccctgatcagccc
cgagtgggtgctgacagccgcccactgtctggaaa
agtcccccagacccagcagctacaaagtgatcctg
ggagcccaccaggaagtgaacctggaacctcacgt
gcaggaaatcgaggtgtccagactgttcctggaac
ccacccggaaggatatcgccctgctgaagctgagc
agccctgccgtgatcaccgacaaagtgattcccgc
ctgcctgcccagccccaactatgtggtggccgaca
gaaccgagtgcttcatcaccggctggggcgagaca
cagggcacatttggagccggcctgctgaaagaggc
ccagctgcctgtgatcgagaacaaagtgtgcaacc
gctacgagttcctgaacggcagagtgcagagcacc
gagctgtgtgccggacatctggctggcggcacaga
tagctgtcagggcgattctggcggccctctcgtgt
gcttcgagaaggacaagtacatcctgcagggcgtg
accagctggggcctgggatgtgccagacctaacaa
gcccggcgtgtacgtgcgcgtgtccagatttgtga
cctggatcgagggcgtgatgcggaacaactga
Exemplary amino acid sequence of human
Lys-Plg
Signal peptide underlined
(SEQ ID NO: 9)
MEHKEVVLLLLLFLKSGQGKVYLSECKTGNGKNYR
GTMSKTKNGITCQKWSSTSPHRPRFSPATHPSEGL
EENYCRNPDNDPQGPWCYTTDPEKRYDYCDILECE
EECMHCSGENYDGKISKTMSGLECQAWDSQSPHAH
GYIPSKFPNKNLKKNYCRNPDRELRPWCFTTDPNK
RWELCDIPRCTTPPPSSGPTYQCLKGTGENYRGNV
AVTVSGHTCQHWSAQTPHTHNRTPENFPCKNLDEN
YCRNPDGKRAPWCHTTNSQVRWEYCKIPSCDSSPV
STEQLAPTAPPELTPVVQDCYHGDGQSYRGTSSTT
TTGKKCQSWSSMTPHRHQKTPENYPNAGLTMNYCR
NPDADKGPWCFTTDPSVRWEYCNLKKCSGTEASVV
APPPVVLLPDVETPSEEDCMFGNGKGYRGKRATTV
TGTPCQDWAAQEPHRHSIFTPETNPRAGLEKNYCR
NPDGDVGGPWCYTTNPRKLYDYCDVPQCAAPSFDC
GKPQVEPKKCPGRVVGGCVAHPHSWPWQVSLRTRF
GMHFCGGTLISPEWVLTAAHCLEKSPRPSSYKVIL
GAHQEVNLEPHVQEIEVSRLFLEPTRKDIALLKLS
SPAVITDKVIPACLPSPNYVVADRTECFITGWGET
QGTFGAGLLKEAQLPVIENKVCNRYEFLNGRVQST
ELCAGHLAGGTDSCQGDSGGPLVCFEKDKYILQGV
TSWGLGCARPNKPGVYVRVSRFVTWIEGVMRNN
Exemplary nucleic acid sequence of human
Midi-Plg
(SEQ ID NO: 10)
ATGGAACACAAAGAAGTGGTGTTGCTCCTGCTGCT
GTTCCTGAAGTCCGGCCAGGGCGATTGCTACCACG
GCGACGGCCAGAGCTACAGAGGCACCAGCAGCACC
ACCACAACCGGCAAGAAGTGCCAGAGCTGGTCCTC
CATGACCCCTCACCGGCACCAGAAAACCCCTGAGA
ATTACCCCAACGCCGGCCTGACCATGAACTACTGT
AGAAATCCCGACGCCGACAAGGGACCCTGGTGCTT
CACAACAGACCCTTCCGTCAGATGGGAATACTGTA
ATCTGAAGAAGTGCAGCGGCACCGAGGCCAGCGTG
GTGGCTCCTCCACCAGTGGTGCTGCTGCCCGATGT
GGAAACCCCCTCCGAAGAGGACTGTATGTTCGGCA
ATGGCAAGGGCTATAGAGGCAAGCGGGCCACCACC
GTGACCGGCACACCTTGTCAGGATTGGGCCGCTCA
GGAACCCCACAGACACAGCATCTTCACCCCAGAGA
CAAACCCTCGGGCCGGACTGGAAAAAAACTATTGT
CGGAATCCTGACGGCGACGTGGGAGGACCTTGGTG
TTATACAACAAACCCACGGAAGCTGTACGATTACT
GTGACGTGCCCCAGTGTGCCGCCCCTAGCTTCGAT
TGTGGCAAGCCCCAGGTGGAACCCAAGAAATGCCC
CGGCAGAGTCGTGGGCGGATGTGTGGCCCATCCTC
ACTCTTGGCCTTGGCAGGTGTCCCTGCGGACCAGA
TTCGGCATGCACTTTTGCGGCGGCACCCTGATCAG
CCCCGAGTGGGTGCTGACAGCCGCCCACTGTCTGG
AAAAGTCCCCCAGACCCAGCAGCTACAAAGTGATC
CTGGGAGCCCACCAGGAAGTGAACCTGGAACCTCA
CGTGCAGGAAATCGAGGTGTCCAGACTGTTCCTGG
AACCCACCCGGAAGGATATCGCCCTGCTGAAGCTG
AGCAGCCCTGCCGTGATCACCGACAAAGTGATTCC
CGCCTGCCTGCCCAGCCCCAACTATGTGGTGGCCG
ACAGAACCGAGTGCTTCATCACCGGCTGGGGCGAG
ACACAGGGCACATTTGGAGCCGGCCTGCTGAAAGA
GGCCCAGCTGCCTGTGATCGAGAACAAAGTGTGCA
ACCGCTACGAGTTCCTGAACGGCAGAGTGCAGAGC
ACCGAGCTGTGTGCCGGACATCTGGCTGGCGGCAC
AGATAGCTGTCAGGGCGATTCTGGCGGCCCTCTCG
TGTGCTTCGAGAAGGACAAGTACATCCTGCAGGGC
GTGACCAGCTGGGGCCTGGGATGTGCCAGACCTAA
CAAGCCCGGCGTGTACGTGCGCGTGTCCAGATTTG
TGACCTGGATCGAGGGCGTGATGCGGAACAACTGA
Exemplary amino acid sequence of human
Midi-Plg
Signal peptide underlined
(SEQ ID NO: 11)
MEHKEVVLLLLLFLKSGQGDCYHGDGQSYRGTSST
TTTGKKCQSWSSMTPHRHQKTPENYPNAGLTMNYC
RNPDADKGPWCFTTDPSVRWEYCNLKKCSGTEASV
VAPPPVVLLPDVETPSEEDCMFGNGKGYRGKRATT
VTGTPCQDWAAQEPHRHSIFTPETNPRAGLEKNYC
RNPDGDVGGPWCYTTNPRKLYDYCDVPQCAAPSFD
CGKPQVEPKKCPGRVVGGCVAHPHSWPWQVSLRTR
FGMHFCGGTLISPEWVLTAAHCLEKSPRPSSYKVI
LGAHQEVNLEPHVQEIEVSRLFLEPTRKDIALLKL
SSPAVITDKVIPACLPSPNYVVADRTECFITGWGE
TQGTFGAGLLKEAQLPVIENKVCNRYEFLNGRVQS
TELCAGHLAGGTDSCQGDSGGPLVCFEKDKYILQG
VTSWGLGCARPNKPGVYVRVSRFVTWIEGVMRNN
Exemplary nucleic acid sequence of
human mini- Plg
(SEQ ID NO: 12)
atggaacacaaagaagtggtgttgctcctgctgct
gttcctgaagtccggccagggcgaggactgtatgt
tcggcaatggcaagggctatagaggcaagcgggcc
accaccgtgaccggcacaccttgtcaggattgggc
cgctcaggaaccccacagacacagcatcttcaccc
cagagacaaaccctcgggccggactggaaaaaaac
tattgtcggaatcctgacggcgacgtgggaggacc
ttggtgttatacaacaaacccacggaagctgtacg
attactgtgacgtgccccagtgtgccgcccctagc
ttcgattgtggcaagccccaggtggaacccaagaa
atgccccggcagagtcgtgggcggatgtgtggccc
atcctcactcttggccttggcaggtgtccctgcgg
accagattcggcatgcacttttgcggcggcaccct
gatcagccccgagtgggtgctgacagccgcccact
gtctggaaaagtcccccagacccagcagctacaaa
gtgatcctgggagcccaccaggaagtgaacctgga
acctcacgtgcaggaaatcgaggtgtccagactgt
tcctggaacccacccggaaggatatcgccctgctg
aagctgagcagccctgccgtgatcaccgacaaagt
gattcccgcctgcctgcccagccccaactatgtgg
tggccgacagaaccgagtgcttcatcaccggctgg
ggcgagacacagggcacatttggagccggcctgct
gaaagaggcccagctgcctgtgatcgagaacaaag
tgtgcaaccgctacgagttcctgaacggcagagtg
cagagcaccgagctgtgtgccggacatctggctgg
cggcacagatagctgtcagggcgattctggcggcc
ctctcgtgtgcttcgagaaggacaagtacatcctg
cagggcgtgaccagctggggcctgggatgtgccag
acctaacaagcccggcgtgtacgtgcgcgtgtcca
gatttgtgacctggatcgagggcgtgatgcggaac
aactga
Exemplary amino acid sequence of human
mini-Plg
Signal peptide underlined
(SEQ ID NO: 13)
MEHKEVVLLLLLFLKSGQGEDCMFGNGKGYRGKRA
TTVTGTPCQDWAAQEPHRHSIFTPETNPRAGLEKN
YCRNPDGDVGGPWCYTTNPRKLYDYCDVPQCAAPS
FDCGKPQVEPKKCPGRVVGGCVAHPHSWPWQVSLR
TRFGMHFCGGTLISPEWVLTAAHCLEKSPRPSSYK
VILGAHQEVNLEPHVQEIEVSRLFLEPTRKDIALL
KLSSPAVITDKVIPACLPSPNYVVADRTECFITGW
GETQGTFGAGLLKEAQLPVIENKVCNRYEFLNGRV
QSTELCAGHLAGGTDSCQGDSGGPLVCFEKDKYIL
QGVTSWGLGCARPNKPGVYVRVSRFVTWIEGVMRN
N
Exemplary nucleic acid sequence of
human micro- Plg
(SEQ ID NO: 14)
atggaacacaaagaagtggtgttgctcctgctgct
gttcctgaagtccggccagggcgcccctagcttcg
attgtggcaagccccaggtggaacccaagaaatgc
cccggcagagtcgtgggcggatgtgtggcccatcc
tcactcttggccttggcaggtgtccctgcggacca
gattcggcatgcacttttgcggcggcaccctgatc
agccccgagtgggtgctgacagccgcccactgtct
ggaaaagtcccccagacccagcagctacaaagtga
tcctgggagcccaccaggaagtgaacctggaacct
cacgtgcaggaaatcgaggtgtccagactgttcct
ggaacccacccggaaggatatcgccctgctgaagc
tgagcagccctgccgtgatcaccgacaaagtgatt
cccgcctgcctgcccagccccaactatgtggtggc
cgacagaaccgagtgcttcatcaccggctggggcg
agacacagggcacatttggagccggcctgctgaaa
gaggcccagctgcctgtgatcgagaacaaagtgtg
caaccgctacgagttcctgaacggcagagtgcaga
gcaccgagctgtgtgccggacatctggctggcggc
acagatagctgtcagggcgattctggcggccctct
cgtgtgcttcgagaaggacaagtacatcctgcagg
gcgtgaccagctggggcctgggatgtgccagacct
aacaagcccggcgtgtacgtgcgcgtgtccagatt
tgtgacctggatcgagggcgtgatgcggaacaact
ga
Exemplary amino acid sequence of human
micro-Plg
Signal peptide underlined
(SEQ ID NO: 15)
MEHKEVVLLLLLFLKSGQGAPSFDCGKPQVEPKKC
PGRVVGGCVAHPHSWPWQVSLRTRFGMHFCGGTLI
SPEWVLTAAHCLEKSPRPSSYKVILGAHQEVNLEP
HVQEIEVSRLFLEPTRKDIALLKLSSPAVITDKVI
PACLPSPNYVVADRTECFITGWGETQGTFGAGLLK
EAQLPVIENKVCNRYEFLNGRVQSTELCAGHLAGG
TDSCQGDSGGPLVCFEKDKYILQGVTSWGLGCARP
NKPGVYVRVSRFVTWIEGVMRNN
Exemplary amino acid sequence of human
Glu-Plg with signal peptide removed
(SEQ ID NO: 16)
EPLDDYVNTQGASLFSVTKKQLGAGSIEECAAKCE
EDEEFTCRAFQYHSKEQQCVIMAENRKSSIIIRMR
DVVLFEKKVYLSECKTGNGKNYRGTMSKTKNGITC
QKWSSTSPHRPRFSPATHPSEGLEENYCRNPDNDF
OGPWCYTTDPEKRYDYCDILECEEECMHCSGENYD
GKISKTMSGLECQAWDSQSPHAHGYIPSKFPNKNL
KKNYCRNPDRELRPWCFTTDPNKRWELCDIPRCTT
PPPSSGPTYQCLKGTGENYRGNVAVTVSGHTCQHW
SAQTPHTHNRTPENFPCKNLDENYCRNPDGKRAPW
CHTTNSQVRWEYCKIPSCDSSPVSTEQLAPTAPPE
LTPVVQDCYHGDGQSYRGTSSTTTTGKKCQSWSSM
TPHRHQKTPENYPNAGLTMNYCRNPDADKGPWCFT
TDPSVRWEYCNLKKCSGTEASVVAPPPVVLLPDVE
TPSEEDCMFGNGKGYRGKRATTVTGTPCQDWAAQE
PHRHSIFTPETNPRAGLEKNYCRNPDGDVGGPWCY
TTNPRKLYDYCDVPQCAAPSFDCGKPQVEPKKCPG
RVVGGCVAHPHSWPWQVSLRTRFGMHFCGGTLISP
EWVLTAAHCLEKSPRPSSYKVILGAHQEVNLEPHV
QEIEVSRLFLEPTRKDIALLKLSSPAVITDKVIPA
CLPSPNYVVADRTECFITGWGETQGTFGAGLLKEA
QLPVIENKVCNRYEFLNGRVQSTELCAGHLAGGTD
SCQGDSGGPLVCFEKDKYILQGVTSWGLGCARPNK
PGVYVRVSRFVTWIEGVMRNN
Exemplary amino acid sequence of human
Lys-Plg with signal peptide removed
(SEQ ID NO: 17)
KVYLSECKTGNGKNYRGTMSKTKNGITCQKWSSTS
PHRPRFSPATHPSEGLEENYCRNPDNDFOGPWCYT
TDPEKRYDYCDILECEEECMHCSGENYDGKISKTM
SGLECQAWDSQSPHAHGYIPSKFPNKNLKKNYCRN
PDRELRPWCFTTDPNKRWELCD!PRCTTPPPSSGP
TYQCLKGTGENYRGNVAVTVSGHTCQHWSAQTPHT
HNRTPENFPCKNLDENYCRNPDGKRAPWCHTTNSQ
VRWEYCKIPSCDSSPVSTEQLAPTAPPELTPVVQD
CYHGDGQSYRGTSSTTTTGKKCQSWSSMTPHRHQK
TPENYPNAGLTMNYCRNPDADKGPWCFTTDPSVRW
EYCNLKKCSGTEASVVAPPPVVLLPDVETPSEEDC
MFGNGKGYRGKRATTVTGTPCQDWAAQEPHRHSIF
TPETNPRAGLEKNYCRNPDGDVGGPWCYTTNPRKL
YDYCDVPQCAAPSFDCGKPQVEPKKCPGRVVGGCV
AHPHSWPWQVSLRTRFGMHFCGGTLISPEWVLTAA
HCLEKSPRPSSYKVILGAHQEVNLEPHVQEIEVSR
LFLEPTRKDIALLKLSSPAVITDKVIPACLPSPNY
VVADRTECFITGWGETQGTFGAGLLKEAQLPVIEN
KVCNRYEFLNGRVQSTELCAGHLAGGTDSCQGDSG
GPLVCFEKDKYILQGVTSWGLGCARPNKPGVYVRV
SRFVTWIEGVMRNN
Exemplary amino acid sequence of human
midi-Plo
(SEQ ID NO: 18)
DCYHGDGQSYRGTSSTTTTGKKCQSWSSMTPHRHQ
KTPENYPNAGLTMNYCRNPDADKGPWCFTTDPSVR
WEYCNLKKCSGTEASVVAPPPVVLLPDVETPSEED
CMFGNGKGYRGKRATTVTGTPCQDWAAQEPHRHSI
FTPETNPRAGLEKNYCRNPDGDVGGPWCYTTNPRK
LYDYCDVPQCAAPSFDCGKPQVEPKKCPGRVVGGC
VAHPHSWPWQVSLRTRFGMHFCGGTLISPEWVLTA
AHCLEKSPRPSSYKVILGAHQEVNLEPHVQEIEVS
RLFLEPTRKDIALLKLSSPAVITDKVIPACLPSPN
YVVADRTECFITGWGETQGTFGAGLLKEAQLPVIE
NKVCNRYEFLNGRVQSTELCAGHLAGGTDSCQGDS
GGPLVCFEKDKYILQGVTSWGLGCARPNKPGVYVR
VSRFVTWIEGVMRNN
Exemplary amino acid sequence of
human mini-Plo
(SEQ ID NO: 19)
EDCMFGNGKGYRGKRATTVTGTPCQDWAAQEPHRH
SIFTPETNPRAGLEKNYCRNPDGDVGGPWCYTTNP
RKLYDYCDVPQCAAPSFDCGKPQVEPKKCPGRVVG
GCVAHPHSWPWQVSLRTRFGMHFCGGTLISPEWVL
TAAHCLEKSPRPSSYKVILGAHQEVNLEPHVQEIE
VSRLFLEPTRKDIALLKLSSPAVITDKVIPACLPS
PNYVVADRTECFITGWGETQGTFGAGLLKEAQLPV
IENKVCNRYEFLNGRVQSTELCAGHLAGGTDSCQG
DSGGPLVCFEKDKYILQGVTSWGLGCARPNKPGVY
VRVSRFVTWIEGVMRNN
Exemplary amino acid sequence of human
micro-Plo
(SEQ ID NO: 20)
APSFDCGKPQVEPKKCPGRVVGGCVAHPHSWPWQV
SLRTRFGMHFCGGTLISPEWVLTAAHCLEKSPRPS
SYKVILGAHQEVNLEPHVQEIEVSRLFLEPTRKDI
ALLKLSSPAVITDKVIPACLPSPNYVVADRTECFI
TGWGETQGTFGAGLLKEAQLPVIENKVCNRYEFLN
GRVQSTELCAGHLAGGTDSCQGDSGGPLVCFEKDK
YILQGVTSWGLGCARPNKPGVYVRVSRFVTWIEGV
MRNN

1. A method for producing plasminogen, the method comprising:
(i) providing a host cell comprising a first recombinant polynucleotide encoding plasminogen and a second recombinant polynucleotide encoding plasminogen activator inhibitor-1 (PAI-1);
(ii) culturing said host cell in a suitable culture medium under conditions to effect expression of plasminogen from the first polynucleotide and PAI-1 from the second polynucleotide.

2. A method of producing recombinant plasminogen, the method comprising the steps of:
(a) providing a first polynucleotide encoding plasminogen,
(b) providing a second polynucleotide encoding PAI-1,
wherein the first and the second polynucleotide are operably linked to a promoter for enabling the expression of the polynucleotides,
(c) providing a host cell,
(d) transforming or transfecting the host cell with the polynucleotides of (a) and (b),
(e) providing cell culture media,
(f) culturing the transformed or transfected host cell in the cell culture media under conditions sufficient for expression of plasminogen and PAI-1, and
optionally (g) recovering or purifying plasminogen from the host cell and/or the cell culture media.

3. The method of claim 1 or 2, wherein the first and second polynucleotides are provided in a single vector.

4. The method of claim 1 or 2, wherein the first and second polynucleotides are provided in separate vectors.

5. The method of claim 4, wherein each vector has a different type of selection marker from the other vector.

6. The method of any one of claims 1 to 4, wherein the method further comprises the step of admixing PAI-1 into the culture media.

7. A method for producing plasminogen, the method comprising:
(i) providing a host cell comprising a recombinant polynucleotide encoding plasminogen;
(ii) culturing said host cell in a suitable culture medium under conditions to effect expression of plasminogen from the polynucleotide,
wherein the culture media comprises PAI-1.

8. A method of producing recombinant plasminogen, the method comprising the steps of:
(a) providing a polynucleotide encoding plasminogen,
wherein the polynucleotide is operably linked to a promoter for enabling the expression of the polynucleotide,
(c) providing a host cell,
(d) transforming or transfecting the host cell with the polynucleotide of (a)
(e) providing cell culture media comprising PAI-1,
(f) culturing the transformed or transfected host cell in the cell culture media under conditions sufficient for expression of plasminogen, and
optionally (g) recovering or purifying plasminogen from the host cell and/or the cell culture media.

9. The method according to any one of claims 1 to 8, wherein the plasminogen is selected from the group consisting of: Glu-Plg, Lys-Plg, Midi-Plg, Mini-Plg and Micro-Plg, as herein described.

10. The method according to any one of claims 1 to 9, wherein the polynucleotide encoding plasminogen comprises, consists or consists essentially of a nucleic acid sequence as set forth in any one of SEQ ID NOs: 1, 5, 6, 8, 10, 12 or 14.

11. The method according to any one of claims 1 to 9, wherein the polynucleotide encoding plasminogen comprises consists or consists essentially of a nucleic acid sequence having at least 75% sequence identity to the sequence set forth in any one of SEQ ID NOs: 1, 5, 6, 8, 10, 12 or 14.

12. The method according to any one of claims 1 to 9, wherein the plasminogen comprises, consists or consists essentially of the amino acid sequence as set forth in any one of SEQ ID NOs: 2, 7, 9, 11, 13, 15, 16, 17, 18, 19 or 20, or has a sequence that is at least 75% identical to the amino acid sequence as set forth in any one of SEQ ID NOs: 2, 7, 9, 13, 15, 16, 17, 18, 19 or 20.

13. The method according to any one of claims 1 to 12, wherein the polynucleotide encoding PAI-1 comprises a nucleic acid sequence of SEQ ID NO: 3.

14. The method according to any one of claims 1 to 12, wherein the polynucleotide encoding PAI-1 comprises, consists or consists essentially of a nucleic acid sequence having at least 75% identity to the sequence set forth in SEQ ID NO:3.

15. The method according to any one of claims 1 to 12, wherein the PAI-1 comprises, consists or consists essentially of the amino acid sequence as shown in SEQ ID NO: 4.

16. The method according to any one of claims 1 to 3, wherein the first and second polynucleotides (i.e., the polynucleotides encoding plasminogen and PAI-1) are provided in a single polynucleotide construct and the single polynucleotide construct comprises, consists or consists essentially of the nucleic acid sequence as shown in SEQ ID NO: 5.

17. The method according to claim 16, wherein single polynucleotide construct comprises a nucleic acid sequence having at least 75% sequence identity to the sequence set forth in SEQ ID: 5.

18. The method according to any one of claims 1 to 17, wherein the host cell is a mammalian cell.

19. The method according to claim 18, wherein the mammalian cell is selected from the group consisting of: Expi293, variants of Expi293, CHO (Chinese Hamster Ovary), HeLa, COS or Vero cells.

20. A vector or nucleic acid construct comprising a first polynucleotide sequence encoding plasminogen and a second polynucleotide sequence encoding PAI-1.

21. The vector or nucleic acid construct according to claim 20, wherein the first and the second polynucleotide are operably linked to a promoter for enabling the expression of the polynucleotides.

22. The vector or nucleic acid construct according to claim 20 or 21, wherein the first polynucleotide sequence encodes a plasminogen selected from the group consisting of: Glu-Plg, Lys-Plg, Midi-Plg, Mini-Plg and Micro-Plg.

23. The vector or nucleic acid construct according to any one of claims 20 to 22, wherein the polynucleotide encoding plasminogen comprises, consists or consists essentially of a nucleic acid sequence as set forth in any one of SEQ ID NOs: 1, 5, 6, 8, 10, 12 or 14.

24. The vector or nucleic acid construct according to any one of claims 20 to 23, wherein the polynucleotide encoding plasminogen comprises, consists or consists essentially of a nucleic acid sequence having at least 75% sequence identity to the sequence set forth in SEQ ID: 1, 5, 6, 8, 10, 12 or 14.

25. The vector or nucleic acid construct according to any one of claims 20 to 23, wherein the plasminogen comprises, consists essentially of, or has an amino acid sequence that is at least 75% identical to the amino acid sequence set forth in any one of SEQ ID NOs: 2, 7, 9, 11, 13, 15, 16, 17, 18, 19 or 20.

26. The vector or nucleic acid construct according to any one of claims 20 to 25, wherein the polynucleotide encoding PAI-1 comprises, consists or consists essentially of a nucleic acid sequence of SEQ ID NO: 3.

27. The vector or nucleic acid construct according to any one of claims 20 to 25, wherein the polynucleotide encoding PAI-1 comprises, consists or consists essentially of a nucleic acid sequence having at least 75% identity to the sequence set forth in SEQ ID NO:3.

28. The vector or nucleic acid construct according to claim 27, wherein the vector encodes a PAI-1 comprising, consisting of or consisting essentially the amino acid sequence as shown in SEQ ID NO: 4.

29. The vector or nucleic acid construct according to claim 20 or 21, wherein the vector comprises a nucleic acid sequence as shown in SEQ ID NO: 5.

30. The vector or nucleic acid construct according to claim 20 or 21, wherein the vector comprises a nucleic acid sequence having at least 75% sequence identity to the sequence set forth in SEQ ID: 5.

31. A host cell comprising a vector or nucleic acid construct of according to any one of claims 20 to 30.

32. An isolated, purified, substantially purified or recombinant plasminogen produced by a method according to any one of claims 1 to 19.

33. An isolated, purified, substantially purified or recombinant plasmin obtained from recombinant plasminogen produced by a method according to any one of claims 1 to 19.

34. A composition comprising plasminogen and plasminogen activation inhibitor isolated, purified or substantially purified from the culture media from a method according to any one of claims 1 to 19.
